(19) United States
(12) Patent Application Publication
(10) Pub. No.: US 2026/0334865 A1
(43) Pub. Date: Oct. 29, 2026

(54) TIME-BASED METHODS AND SYSTEMS FOR THE ASSESSMENT AND PREDICTION OF RECOVERY FROM PHARMACOLOGIC NEUROMUSCULAR BLOCKING AGENTS

(71) Applicant: Robert E. Minahan, Brookeville, MD (US)

(72) Inventor: Robert E. Minahan, Brookeville, MD (US)

(21) Appl. No.: 19/649,941

(22) Filed: Apr. 16, 2026

Related U.S. Application Data
(60) Provisional application No. 63/795,902, filed on Apr. 28, 2025.

(51) Int. Cl. A61B 5/00; A61B 5/395; A61B 5/397; G16H 20/10

(52) U.S. Cl. CPC A61B 5/4821; A61B 5/395; A61B 5/397; G16H 20/10

(57) ABSTRACT

A device and methods for estimating the degree of pharmacologic neuromuscular blockade (NMB), predicting muscular status with full recovery, and predicting time to recover to specified degrees of neuromuscular blockade including full recovery. An embodiment utilizes techniques of electrical repetitive stimulation of a motor nerve with recording of the resulting contraction from its innervated muscles. The suppressive effect of NMB is measured and examination of a limited subset of recorded NMB recovery data is utilized to create a predictive map of the entire NMB recovery pattern over time using pharmacologic principles. The so-created predictive map allows estimation of relevant measures of NMB at any timepoint and NMB dosing guidance.

### CROSS-REFERENCE TO RELATED APPLICATIONS

This application claims priority from U.S. Provisional Patent Application Ser. No. 63/795,902 entitled “Time-based Methods and Systems for the Assessment and Prediction of Recovery from Pharmacologic Neuromuscular Blocking Agents” and filed on Apr. 28, 2025, the disclosure of which is incorporated herein by reference in its entirety.

### TECHNICAL FIELD

Embodiments of the present disclosure relate to medical devices and methods of operating these devices for assessment of the functional status of the neuromuscular junction (NMJ) in the presence of pharmacologic neuromuscular blocking agents. Such devices allow the measurement of muscle relaxation induced by neuromuscular blockade which, in turn, promotes adequate degrees of relaxation needed to perform procedures and allows identification of adequate recovery when muscle function is required.

### BACKGROUND

### Discussion of Related Art

Administration of a nondepolarizing neuromuscular blockade (NMB) agent is commonly utilized during medical procedures or in the intensive care unit in situations where muscle relaxation facilitates clinical goals. When utilized, NMB administration requires some form of measurement of the NMB effect in order to guide its dosing and track its recovery. Improvement in measurements used to describe the degree of relaxation and the ability to predict future behavior of the NMB holds clinical interest.

The measurement of the effect of NMB requires some understanding of interaction of the endogenous neurotransmitter acetylcholine (ACh) and the pharmacologically administered NMB agent. Under normal conditions, depolarization of a motor nerve releases ACh into the neuromuscular junction and the ACh then binds to receptors on the muscle and evokes contraction. When NMB is also present at the neuromuscular junction, the NMB agent competes with the ACh for binding to the muscle's receptors, thereby reducing or eliminating a response at the muscle. Overall, it is the relative concentration of ACh to NMB that will determine the degree of a muscle's response to nerve stimulation.

Measurement of NMB is clinically performed via examination of muscle responses following a motor nerve activation. Measures may directly assess the suppressive effects of NMB (“direct measures”) by comparing the size of muscle response to its expected size in the absence of NMB. Other measures indirectly predict the suppressive effect of NMB by examining the muscle's response to repetitive nerve stimulation (“indirect measures”). Repetitive nerve stimulation is associated with a reduced amount of ACh released from the motor nerve upon each subsequent stimulation which in turn changes the relative concentrations of ACh and NMB at the neuromuscular junction. When sufficient NMB is present, repetitive nerve stimulation results in a series of muscle responses of diminishing size. The phenomenon of diminishing muscle responses in response to repetitive nerve stimulation (“fade”) is a necessary part of the measurement of all commonly used indirect measures and some direct measures. One commonly used rubric for repetitive nerve stimulation is the “train-of-four” (TOF) stimulation where an electrical stimulus depolarizes a motor nerve four times in succession to produce fade. Measurement of fade under this rubric is made via the train-of-four ratio (TOFr) where the TOFr is represented by the ratio of the quantitated fourth muscle response relative to the first muscle response.

The TOFr is an indirect measure that is convenient in that it can give an indication of a degree of NMB using only single time point data. The TOFr validity as a measure of NMB is supported by correlation to a direct measure, the T1%, across a portion of the spectrum of NMB depth. The T1% is the ratio of a muscle's response in the presence of NMB relative to a baseline response in the same muscle previously obtained in the absence of NMB. Of note, the T1% gives insight into the degree of NMB across a wide spectrum, whereas the TOFr is only correlative to the T1% at relatively low degrees of NMB. At the same time, the T1% has its own drawbacks in that the required baseline response may be clinically inconvenient to obtain and thus is often not available, the measure is dependent on correct acquisition of the baseline, and the baseline measure is not a reliably stable indicator of full recovery over time with routine underestimation of NMB depth occurring.

In addition to tracking the effect of NMB on a muscle's response, the pharmacology of the NMB agent in the body is also tracked. While the amount of ACh released with each successive stimulus of the TOF decreases, the amount of ACh released by each of the component ordinal stimulus of the TOF remains effectively constant over time as long as no external influence is applied. The NMB concentration, on the other hand, decreases over time as it is eliminated by the body following a single dose. As a result, the TOF muscle responses increase over time with the largest first response of the TOF fully recovering first and the smallest fourth response recovering last. The graphical shape of an ordinal muscle response recovery vs. time follows a sigmoidal pattern which is well known in the art and applies to both the first ordinal muscle response to repetitive nerve stimulation as well as the later ordinal responses. Unfortunately, the pharmacologic behavior following NMB administration is complex and despite the availability of many proposed models, efforts to reliably predict NMB concentrations have failed to produce a comprehensive model. Additionally, there is significant interindividual variation in the rates of elimination of NMB from the body further complicating pharmacologic modeling of NMB effects.

Given its use as a standard assessment tool, attempts have been made to model and predict the recovery of the TOFr. For example, U.S. Pat. No. 11,813,053 (Batsman et al.) discloses a method to utilize the recovery trend of the TOFr as well as that of the train-of-two ratio (ratio of the second muscle response to the first) to predict subsequent recovery of the TOFr. The prediction process is begun once these ratios begin to rise and the shape of the earlier recovering train-of-two ratio is used to predict the shape of the later TOFr. The predictive process is aided by use of population data to suggest a typical curve recovery path based on early trends of recovery.

U.S. Patent Application Publication No. 2019/0223764 (Hulvershorn et al.) discloses a method to predict timing of recovery of the TOFr to a chosen value based on extrapolation of prior values for the specific patient or via data for patients with similar clinical characteristics.

U.S. Patent Application Publication No. 2009/0327204 (Gilhuly) discloses a method to control administration of NMB agents using the predicted effect of NMB dosing based on mathematical modeling of a generally expected effect of the NMB dose followed by adjustment of predictions based on the deviation from the model using the observed behavior of the TOFr.

The prediction strategies described above suffer from several disadvantages. First, all rely on the observed recovery pattern in the TOFr. While these efforts may provide some insight, the initiation of recovery of the TOFr is a late event relative to their component muscle responses because the TOFr only begins a clear trend of ascent after the fourth response is not only recovering, but recovering proportionately faster than the first response. The importance of earlier estimation with any prediction method cannot be overemphasized. First, there is the advantage that earlier prediction allows earlier impact on clinical decision making. This impact is magnified when considering that NMB is typically administered using intermittent redosing to keep the NMB within a desired therapeutic range. Under this rubric, only partial cycles of recovery will be available for analysis prior to a point where the NMB is allowed to fully recover. Prediction methods using relatively late recovery events are less likely to capture sufficient portions of the recovery process during this cycling of NMB levels and the late-recovering TOFr is unlikely to capture this while NMB is in its therapeutic range.

For Hulvershorn et al., there is lack of detail in methods describing how predictions of TOFr future behavior are made beyond extrapolating a current trend or use of large group data to suggest an average path. Batsman et al. has a similar strategy with the addition of the marginally earlier predictions from use of the modeling of the train-of-two ratio. Finally, Gilhuly has a detailed method using specific pharmacologic modeling models prior to corrections using observed data. The use of pharmacologic modeling of NMB concentrations has the potential to enhance the prediction of the effect of NMB; however, this is hampered by use of the TOFr as the effect measured because, in addition to its late recovery, the derived measure of two different effects (first and fourth muscle response recovery) adds unnecessary complexity and skew to the analysis. Finally, this method's reliance on a specific model may falter if the model is not sufficiently robust which has been the case for models proposed to date.

### SUMMARY

It is an aspect of a present invention embodiment to characterize the behavior of a muscle's response to repetitive nerve stimulation during recovery from NMB. To this purpose, the sequence of ordinal compound motor action potentials (CMAPs) resulting from repetitive nerve stimulation are quantified and recorded with respect to elapsed time. This process creates a series of recovery curves corresponding to the ordinal CMAPs labeled herein for the TOF as C1, C2, C3, and C4.

In one embodiment, characterization of the CMAP recovery curves is based on identifying definable points (“landmarks”) on each recovery curve. Identification of a small subset of available landmarks (e.g., two to three) on one or more of the ordinal recovery curves is used to predict the remaining landmarks. Once landmarks are estimated for each curve, contiguous curves can be estimated, thus creating a recovery map which in turn is used to predict the timing and ordinal CMAP quantification at any point of interest.

In an embodiment, landmarks are associated with specific NMB concentrations in the body which are then used to characterize the NMB elimination rate specific to the individual patient. Once the elimination rate is estimated, standard pharmacokinetic principles are applied to predict future NMB concentrations over time. Then, using standard pharmacokinetic/pharmacodynamic techniques, NMB concentration can, in turn, be used to mathematically predict the timing of CMAP growth over all future (and past) time points. As a result, another form of the NMB recovery map is created.

In an embodiment, plots of data are produced that, according to pharmacological principles, are expected to be linear. Once linear behavior is identified, the parameters of the line are used to predict all CMAP amplitudes over time. As a result, another form of the NMB recovery map is created.

In an embodiment, disruption from the expected ongoing NMB recovery course (deviation from the recovery map) is detected and characterized. In one implementation, an abrupt alteration of signals is analyzed to identify administration of an NMB agent, administration of an NMB reversal agent, detection of another direct impactor of neuromuscular function, and/or disruption of the normal data acquisition process. When a major alteration is detected, the recovery analysis is paused and, when recovery resumes, a determination is made as to whether information from prior recovery maps remains valid or if a new analysis is required. In another implementation, gradual deviation of the ongoing recovery may occur due to factors that modulate the function of the neuromuscular junction (e.g., alteration of anesthetics, administration of other drugs that impact neuromuscular junction function, or patient systemic effects). These impactors are incorporated into the recovery pattern through a continually updating/optimizing process typically without a pause in analysis.

In an embodiment, a measurement of the effect of NMB is provided. In one implementation of this embodiment, the quantity to which CMAPs recover after the effect of NMB has waned is predicted as part of the recovery map creation process. This value, “Cmax” is then used to identify the proportionate degree of recovery for each of the ordinal CMAPs at the time of assessment (current quantification for each ordinal CMAP relative to Cmax). These measures, and derivatives thereof, provide a direct indication of the degree of NMB at the time of data acquisition which are, in turn, used to gauge the effect of NMB on other clinically relevant muscle functions impacted by NMB. In another implementation, a broader description of the stage of NMB recovery is described using the relative recovery patterns and recovery rates of each ordinal CMAP relative to the others. In another implementation, the measurement of NMB across different muscles can be assessed such that known differences in responses to NMB are characterized such that measurement in one muscle predicts the simultaneous activity in all other muscles that have been appropriately characterized for this purpose.

The above and still further aspects, features, and advantages of the present invention will become apparent upon consideration of the following detailed description of example embodiments thereof, particularly when taken in conjunction with the accompanying drawings.

### BRIEF DESCRIPTION OF THE DRAWINGS

FIG. 1 is a block diagram of a neuromuscular blockade assessment device according to an embodiment of the present invention.

FIG. 2A-2D illustrate graphical representations of CMAP responses to TOF repetitive nerve stimulation with responses to stimuli displayed with voltage on the Y-axis and time on the X-axis according to an embodiment of the present invention.

FIG. 3A-3B illustrate graphical representations of CMAP recovery patterns from NMB according to an embodiment of the present invention.

FIG. 4A-4B illustrate graphical representations of recorded data points of CMAP amplitude vs. time resulting in sigmoid curves for the first, second, and fourth compound motor action potentials from the TOF stimulation (“C1”, “C2”, and “C4”) according to an embodiment of the present invention.

FIG. 5 illustrates a flowchart of a method of measuring and predicting NMB recovery according to an embodiment of the present invention.

FIG. 6A-6D illustrate graphical and tabular representations of predictions of recovery with differing amounts of data available for evaluation according to an embodiment of the present invention.

FIG. 7 illustrates a graphical representation of stages of recovery based on a relative recovery status of each CMAP assessed according to an embodiment of the present invention.

FIG. 8 illustrates a graphical representation of simultaneous recovery in two distinct muscles according to an embodiment of the present invention.

FIG. 9 is a block diagram of an example computing device according to an embodiment of the present invention.

### DETAILED DESCRIPTION OF EXAMPLE EMBODIMENTS

Administration of NMB during surgical procedures relaxes muscles which, in turn, may improve surgical access, improve ventilation, and prevent unwanted and potentially hazardous patient movement. During a medical procedure, the targeted degree of muscle relaxation from NMB may vary for a number of reasons including the stage of the procedure, the need for an ability to assess muscle function, or the need for the patient to recover to the point that they can breathe on their own upon removal of external ventilation. As such, there is utility in both being able to measure the degree of NMB at any given time and to predict the timing of future degrees of NMB should the body's clearance of the NMB agent be allowed to proceed without further NMB agent administration. A present invention embodiment provides a device to assess recovery from nondepolarizing neuromuscular blocking agents that meets both the measurement and prediction goals for the degree of NMB. Various embodiments of the present invention are given in this description and in the drawings which are meant to serve as examples and not be limiting or meant to serve as preferred over other embodiments.

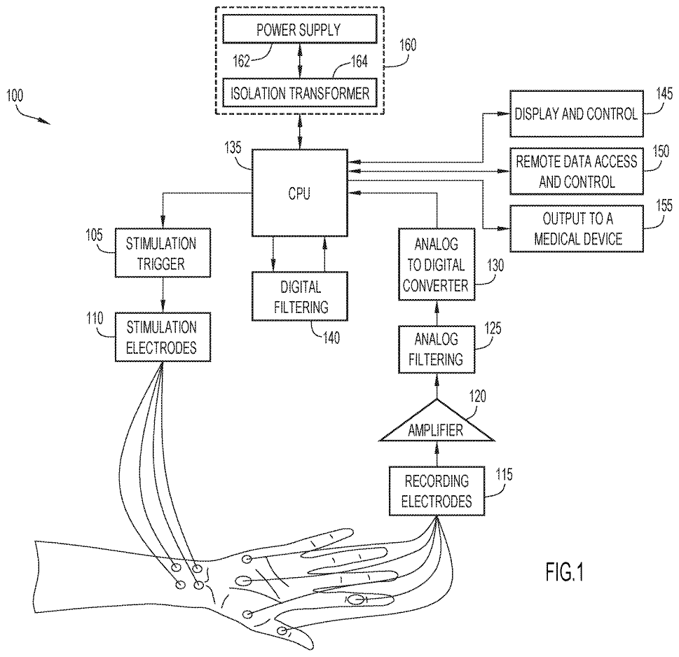

FIG. 1 illustrates a device 100 for electrical assessment of NMB according to an example embodiment. Device 100 may assess NMB during a surgical or other medical procedure to adjust administration of NMB or other agents. Device 100 includes a stimulation output trigger 105 that generates electrical pulses that are delivered at the desired current (e.g., 5-100 mA), voltage (e.g., 5-300V), and pulse width (e.g., 50-1000 μs). Stimulation pulses are delivered individually or in a repetitive series (e.g., 1-250 pulses) with the repetitive pulses delivered at a desired frequency (e.g., 0.1-50 Hz). The stimulation trigger delivers the chosen electrical output to the targeted nerves via stimulation electrodes 110 attached to the body (e.g., arm, wrist, and/or hand as viewed in FIG. 1) in proximity to targeted motor nerves. The stimulation electrodes may be attached to the surface of the skin via conductive adhesive or may be transcutaneous electrodes (e.g., needles).

As an example, a common stimulation technique is a train-of-four (TOF) repetitive nerve stimulation. In this rubric, a series of four pulses is delivered at approximately 2 Hz. A desired intensity of stimulation is achieved via the combination of current (or voltage) and the pulse width. The typical stimulation intensity is supramaximal with supramaximal defined as a level of stimulation that activates all motor axons that contribute to a resulting compound motor action potential. In one implementation, the stimulation level is targeted to 10% above the supramaximal threshold where the supramaximal threshold is defined as the minimum stimulation that produces a maximal CMAP.

Stimulation electrodes are typically used in pairs with one electrode negatively charged (cathode) and one electrode positively charged (anode). The paired electrodes may both be placed along the course of the nerve (e.g., bipolar electrode configuration) with preferred arrangement placing the cathode distal on the nerve relative to the more proximal anode. Alternatively, a monopolar configuration may be used in which the cathode is placed close to the nerve and the anode is displaced from the expected anatomical course of the nerve. The typical techniques do not preclude other stimulation techniques and, for example, a monopolar electrode configuration may be preferred for deep nerves and submaximal stimulation may be preferred in certain settings. Furthermore, a mixed polarity pulse may be delivered (e.g., biphasic) in which the one electrode is first the cathode and then the anode with the opposite sequence in the other electrode.

Initiation of the delivery of a chosen set of stimulation pulses (e.g., TOF) is restricted to periods where the nerve has not been previously activated in a time frame that would alter the results of the intended testing. The duration of this time period for testing exclusion may vary by the preceding type of activation (e.g., <5 hz vs. ≥20 hz) with the typical exclusion period for prior low frequency stimulation being ten seconds. Device 100 is programmed to restrict delivering separate trains of pulses within the chosen exclusionary window. In addition, sensing is utilized to also exclude windows after nerve activation from outside sources (e.g., from intraoperative neuromonitoring testing separate from the primary device). To this end, device 100 senses stimulation artifact and muscle activation via recording electrodes 115 and then delays initiation of stimulation until an appropriate window is present. Similarly, this sensing also delays stimulation in the presence of sensed excessive electrical artifact of a degree that might impair correct CMAP quantification (e.g., from electrocautery). Finally, the results of testing are rejected if electrical artifact or external nerve activation intrudes in the midst of a stimulus train delivery with testing resumed after an appropriate delay following cessation of the confounding activity. In addition to examination of the recorded electrical activity, in another implementation, detection of potentially confounding activity is augmented via detection from a range of techniques including, but not limited to: output triggers from other electrical devices, detection of confounding device activation at/near its source (e.g., electromagnetic detection of activation via sensors placed on electrocautery wires), mechanical sensors of muscle movement, or videography.

Upon successful delivery of a train of pulses, the associated motor nerve depolarizes, the wave of depolarization propagates to the neuromuscular junction, and ACh is released into the neuromuscular junction cleft. ACh then diffuses within the cleft and binds to receptors on the muscle. If a sufficient amount of ACh is able to bind, an action potential in the muscle fiber is triggered causing muscle contraction. The preferred recording technique for identifying muscle activity is EMG (e.g., electromyography) with the summated electrical activity of muscle activation recorded as a CMAP. The recorded electrical activity of the CMAP detected by recording electrodes 115 is amplified by an amplifier 120, filtered to the relevant frequency spectrum of the CMAP by analog filters 125, and undergoes analog to digital conversion by an analog to digital converter 130. These components may be implemented by any conventional or other amplifiers, filters, and converters. The digitized data is delivered to a CPU 135 or other computing device where electrical signals may be further filtered by conventional or other digital filtering 140, and CMAPs are identified, quantified and undergo further analysis according to the descriptions given below. Data review, configuration of acquisition parameters, and user input of clinical information are made through a device interface or console 145. Operations at the device console include adjustment of stimulation parameters, adjustment of signal analysis, review of CMAP waveforms, review of all relevant numerical data, predictions of timing for future recovery events, and directives related to patient dosing. User input regarding medication dosing or other patient data may be entered through this console or may be directly downloaded from the patient's electronic medical record to the CPU. A separate remote data access and control device 150, such as a computer or smart phone, may also be utilized which performs the same functions as the device console and may have additional configuration capabilities. The information may be used by medical personnel to adjust administration of NMB or other agents.

CPU 135 also controls or provides information (or controls) to other medical devices 155, such as a drug infusion device to control administration of NMB, reversal agents, etc. or a neuromonitoring system to control nerve stimulation and other monitoring aspects. Finally, data obtained is stored for future use and review. All contiguous device components are powered by a power source 160 including an isolated power supply 162 and transformer 164. These may be implemented by any conventional or other components.

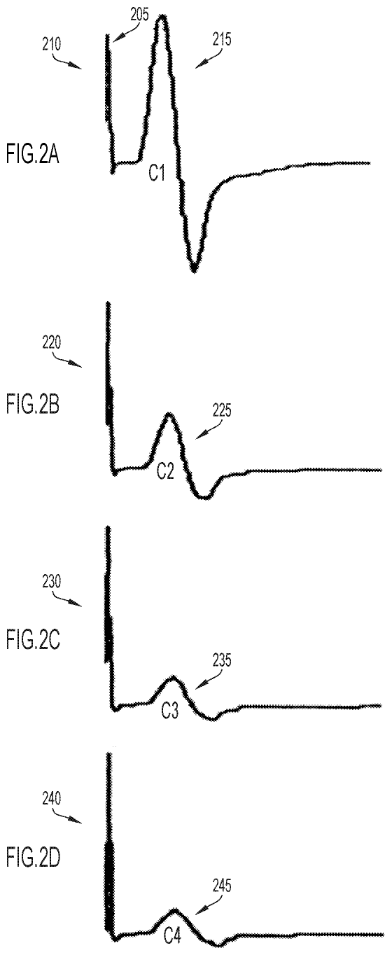

FIG. 2A-2D illustrate graphical representations of the ordinal CMAPs following a TOF stimulation. FIG. 2A illustrates a graphical representation 210 of the muscle's electrical activity following the first stimulation pulse of the TOF using voltage (Y-axis) over time (X-axis). An initial spike 205 is present representing a stimulation artifact followed sequentially by a return to baseline, a peak, a trough, and then a gradual return to baseline. CMAP 215 is the electrical activity between the initial deflection from baseline and the eventual return to baseline with amplitude determined by the vertical difference between the peak and the trough. FIGS. 2B-2D illustrate similar graphical representations 220, 230, and 240 of the electrical activity following the second, third, and fourth pulses of the TOF respectively with associated CMAPs 225, 235, and 245. A characteristic decremental pattern of response (“fade”) in the presence of NMB is seen with CMAP amplitudes becoming progressively smaller with each subsequent stimulus.

The CMAPs of FIGS. 2A-2D are also labeled, in order of stimuli, as C1, C2, C3, and C4 according to an embodiment of the present invention. Furthermore, the notation “Cx” is used herein to refer to any or all of the ordinal CMAPs (e.g., C1, C2, C3, and/or C4 for TOF stimulation). Depending on the context, the C1, C2, C3, and C4 nomenclature may refer to both the associated CMAP or its quantified value. This “C” notation is analogous to, but distinct from, prior work where T1, T2, T3, and T4 nomenclature is used to denote muscle responses to TOF stimulation in which the “T” nomenclature refers to muscle twitches and reflects work that is more centered on mechanical muscle responses. The “C” nomenclature, on the other hand, refers to the CMAP and is more applicable to EMG recording where CMAPs are the recorded response of interest with CMAPs routinely recorded in the absence of a measurable mechanical response (twitch).

In a preferred embodiment, TOF stimulation is the repetitive nerve stimulation technique, electrical (EMG) recording is the muscle response gathering technique, and CMAP amplitude is the CMAP quantification metric. In another implementation, the area under the curve for each CMAP is the quantification metric for the muscle's response. The preferred techniques do not preclude other techniques not specified in the preferred parameters, including numbers of stimulations in the repetitive stimulation delivery or the type of recording to include mechanomyography, acceleromyography, and kinemyography in which cases the recorded movements are not CMAPs and are more commonly referred to as twitches.

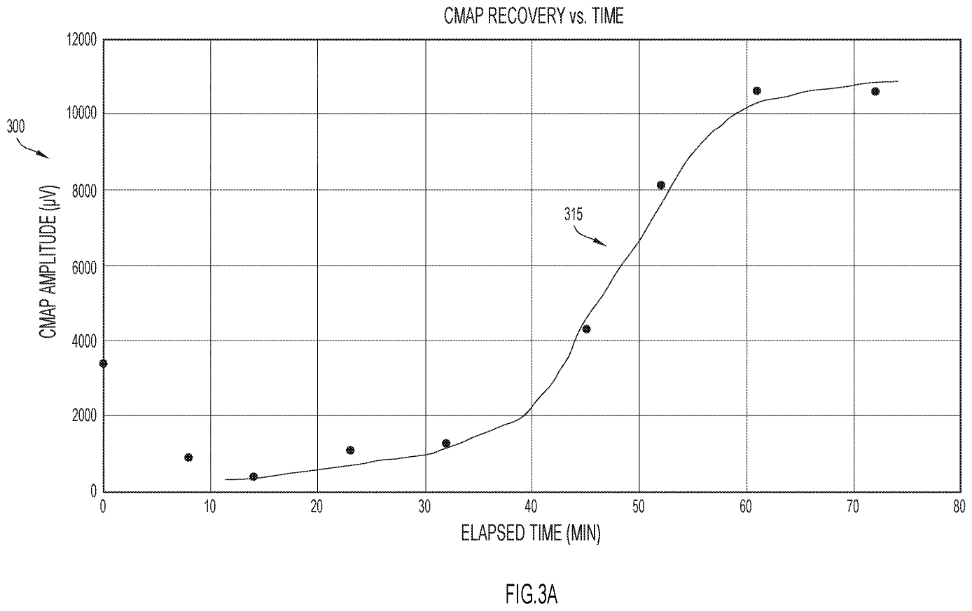

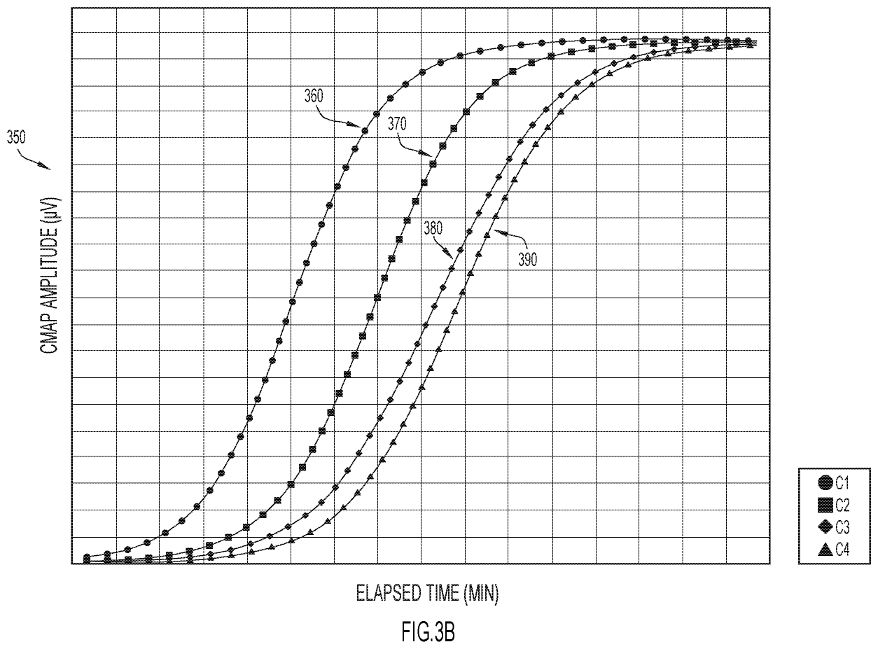

FIG. 3A illustrates a graphical representation 300 of CMAP amplitude (Y-axis) over time (X-axis) with a single CMAP recovery curve 315 illustrating the recovery of CMAP amplitudes as NMB is allowed to wane. Graphical representation 300 shows a plot of recovery curve 315 for a single ordinal CMAP amplitude (in μV) vs time (in minutes). Discrete recorded data points represent the recorded data for this patient while the contiguous curve represents the expected course of the recovery based on the recorded points. For the purposes of an embodiment of the present invention, the components of this sigmoid curve are separated into phases with the early, relatively low-slope portion of the curve on the left of the plot considered as an early phase, the middle rapid rise portion as a rapid phase, and the late relatively low-slope component as a plateau phase.

Once recovery is initiated, the CMAP's amplitude vs. time follows a course of progressive increase as long as recovery is allowed to proceed along its typical course. Absent outside influence, the progressive increase in responses represents a mostly constant neuromuscular junction concentration of the agonist (ACh) for any given ordinal CMAP while the NMB concentration wanes over time due to the body's clearance of the drug. Relative concentrations of ACh associated with each of the ordinal CMAPs are progressively lower with each of the ordinal CMAPs resulting from TOF stimulation (e.g., C1 highest concentration, C4 lowest concentration). In a preferred embodiment, when the slope of a recovery curve is consistently positive it is assumed that recovery is in progress and analysis can proceed. This point is demonstrated in FIG. 3A with the onset of recovery noted soon after the ten-minute mark. Prior to the onset of recovery, the displayed curve 315 also shows a common sequential scenario in which after a sufficient dose of NMB, CMAP amplitudes initially fall corresponding to administration of NMB agent and equilibration of body's NMB concentration levels (points prior to the ten-minute mark). As equilibration progresses, CMAP amplitudes reach a nadir which is then followed by progressively rising amplitudes with the curve showing the consistently positive slopes denoting recovery. FIG. 3B illustrates a graphical representation 350 of CMAP amplitude (Y-axis) over time (X-axis) for a set of recovery curves 360, 370, 380, and 390 for all ordinal CMAPs of the TOF in a single subject. Graphical representation 350 shows a representative example of all ordinal CMAP recovery curves for TOF stimulation through the full course of recovery. Recovery occurs in sequence with curve 360 (e.g., C1 curve as viewed in FIG. 3B) to the left (highest concentration of associated ACh), curve 390 (e.g., C4 curve as viewed in FIG. 3B) furthest to the right (lowest concentration of associated ACh), and curves 370 (e.g., C2 curve as viewed in FIGS. 3B) and 380 (e.g., C3 curve as viewed in FIG. 3B) intermediate in both timing and ACh concentrations.

Curve 360 (e.g., C1 curve as viewed in FIG. 3B) is associated with the highest neuromuscular junction concentrations of ACh relative to the other ordinal CMAPs and, thus, at each time point where a TOF stimulation is delivered, it will have the largest CMAP amplitude. Likewise, curve 390 (e.g., C4 curve as viewed in FIG. 3B) is associated with the lowest concentration of ACh and it will have the lowest CMAP amplitude at each time point with curves 370 (e.g., C2 curve as viewed in FIGS. 3B) and 380 (e.g., C3 curve as viewed in FIG. 3B) being intermediate between the C1 and C4 curves (e.g., curves 360 and 390 as viewed in FIG. 3B). It follows that the C1 curve is the first to initiate recovery and the C4 curve is the last to complete recovery with the full set of data showing four side-by-side sigmoidal curves for the ordinal CMAPs as displayed. FIG. 3B also demonstrates characteristic close approximation of the C3 and C4 curves which may limit the amount of distinct information that one of these curves may deliver over the other. In one embodiment, all available ordinal CMAPs are used in data analysis. In another embodiment, one or more of the ordinal CMAPs are excluded from the initial data analysis (e.g., typically C3 being the one excluded). In all embodiments in which an ordinal CMAP is not utilized for initial analysis, it remains available as a comparison value to confirm validity of, and findings made from, the utilized CMAPs.

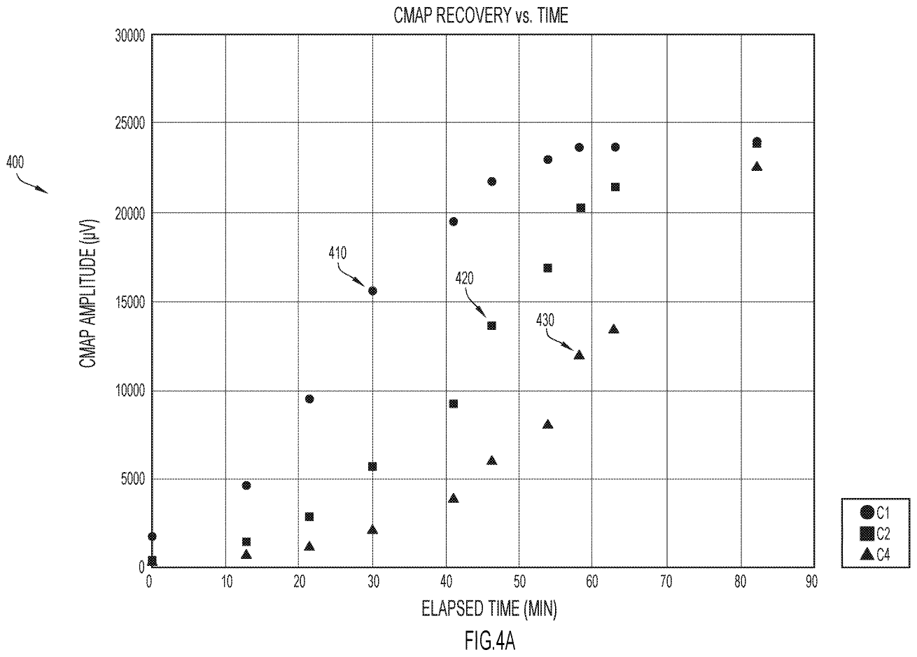

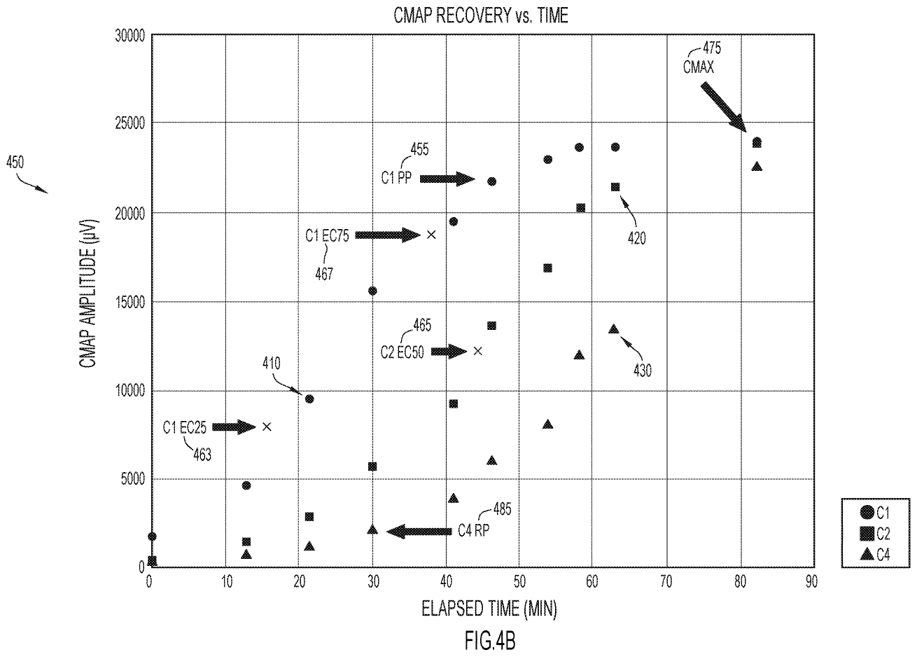

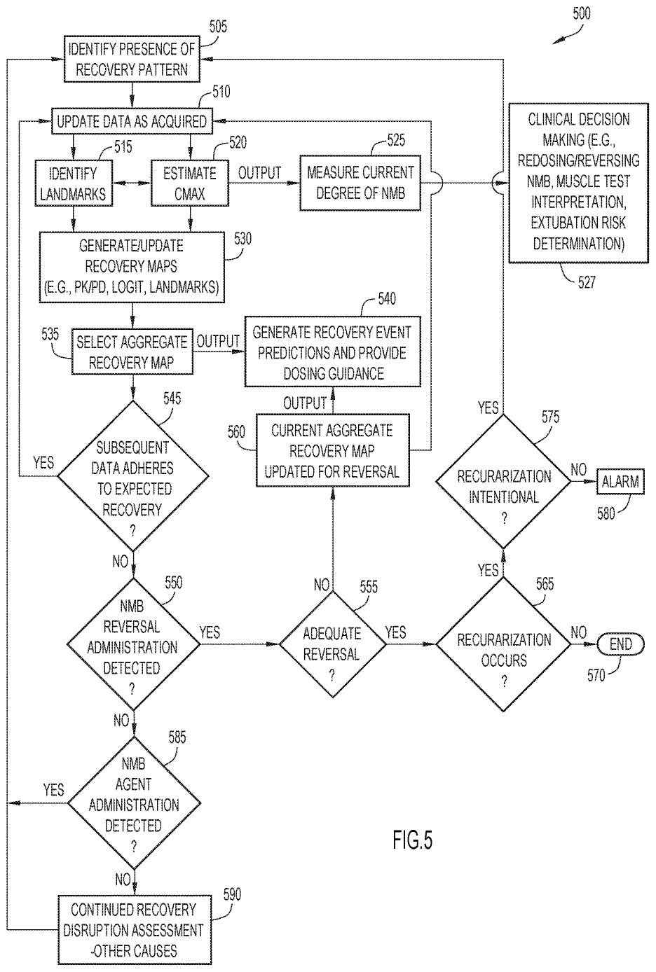

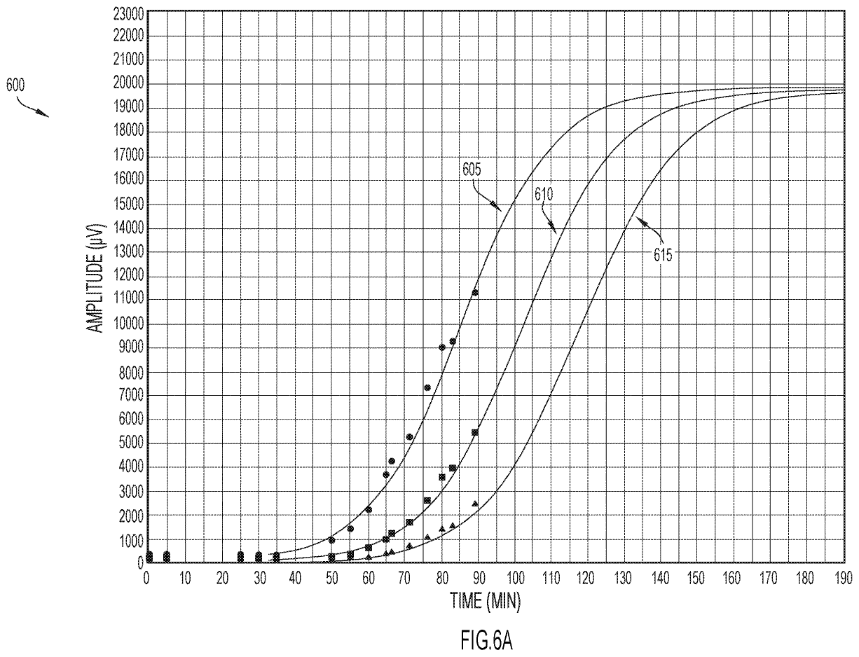

FIG. 4A illustrates a graphical representation 400 of CMAP amplitude (Y-axis) over time (X-axis) for data points indicating underlying curves 410 (corresponding to C1), 420 (corresponding to C2), and 430 (corresponding to C4) with the underlying sequential and sigmoidal nature of the recovery of each CMAP again demonstrated using periodic data. Graphical representation 400 shows observed data points for C1, C2, and C4 CMAP curves over an interval comprising much, but not all, of a recovery cycle. The underlying sigmoidal shapes and sequential pattern of the ordinal CMAPs are observed. Stereotyped points of interest on each of the underlying continuous ordinal recovery curves may be defined/identified (“landmarks”). In a preferred embodiment, landmarks serve to denote points in recovery where the nicotinic receptor occupancy of an NMB agent is adequately preserved across a population and thereby allows linkage of observed data to the underlying pharmacological processes.

FIG. 4B shows a graphical representation 450 with the same data as in FIG. 4A, but with selected landmarks identified. An early landmark may be identified as the point where the recovery curve transitions from early to rapid phase, and may be referred to as, by way of example, the Recovery Point (RP). This point is estimated for C4 (e.g., point 485 (or C4 RP) as viewed in FIG. 4B). Another landmark is represented by the point where the CMAP amplitude reaches half its maximal recovery value, and may be referred to, by way of example, using its associated effective concentration for a 50% effect (EC50). This point is estimated for C2 at a point between recorded data points (e.g., point 465 (or C2 EC50) as viewed in FIG. 4B). Additional landmarks may be defined based on their proportion of maximal value and, by way of example, the point representing 25% recovery (e.g., point 463 (or C1 EC25) as viewed in FIG. 4B) and the point representing 75% recovery (e.g., point 467 (or C1 EC75) as viewed in FIG. 4B) are estimated. Another landmark is represented by the point where the CMAP recovery curve transitions from rapid phase to plateau phase, and may be referred to, by way of example, as the Plateau Point (PP). This point is indicated for the C1 recovery curve (e.g., point 455 (or C1 PP) as viewed in FIG. 4B). Two additional landmarks corresponding to the onset of recovery (ON) and the offset of recovery (OFF) are not indicated due to the absence of data points that clearly demarcate them. Landmarks are identified by estimating their CMAP amplitude and their time of occurrence on the CMAP recovery curve plot of amplitude over time.

The maximal CMAP quantity that recovery curves reach upon full recovery from NMB is an important quantity for a number of purposes including landmark identification. This quantity may be referred to, by way of example, as Cmax and this quantity is represented, by way of example, in FIG. 4B as the amplitude associated with both C1 and C2 at point 475. In a preferred implementation, Cmax is the full recovery CMAP amplitude. In another implementation, Cmax is the full recovery CMAP area under the curve.

In one embodiment, landmarks are selected as points where CMAP quantities reach a defined proportion of Cmax with examples including ON, EC25, EC50, EC75, and OFF. Landmarks defined by proportionate recovery have estimated amplitudes determined by the product of the specified proportion and the estimated Cmax. In one embodiment, the time of proportional landmarks is determined via imputation between existing data points. In another embodiment, the time of the landmark is determined by extrapolation from existing data points. In one implementation, linear extrapolation is used to estimate time, particularly within the rapid recovery phase (e.g., within proportions of 25%-75%). In another implementation, adjustment for expected curvature is made by using a population average curve for the targeted segment of the curve (e.g., for targeted proportions <25% or >75%). In another implementation, the steepness of the population average curve is refined via adjustment of the Hill coefficient based on the observed maximal slope.

In another embodiment, landmarks are defined by characteristics of the slope of the recovery curve with examples including RP and PP. In one implementation, landmarks are defined as the point at which the slope of the recovery curve reaches a specified proportion of the maximal recovery curve slope. In another implementation, landmarks are localized to the points where the maximal rate of change in slope occurs. In one implementation, slope change maxima are determined by solving the third derivative of equations for recovery map creation (described herein) when equated to 0 in order to find the associated time (e.g., logit map) or the associated concentration (e.g., PK/PD map). In one embodiment, the RP (e.g., point 485 (or C4 RP) as viewed in FIG. 4B) and the PP (e.g., point 455 (or C1 PP) as viewed in FIG. 4B) are determined by the points where the CMAP slope reaches its greatest rate of change. In an embodiment, RP and PP are determined by the point where the slope, expressed as a proportion of the maximal slope, best predicts the point where the CMAP slope reaches its greatest rate of change. In another embodiment, additional landmarks are determined based on their slope relative to the maximal slope.

Landmark identification may be aided by additional analyses, some of which are common to all landmarks and some of which are specific to the landmark. In general, amplitudes of points on one recovery curve approximate the amplitude on the others and can be used as a reasonable approximation for determining landmark location on other curves. Similarly, when the time differences between recovery curves are known for one or more points, this time difference can be used to estimate timing of a landmark on one recovery curve when the analogous landmark is identified on the other recovery curve. Also, NMB effective concentration or NMB occupancy of nicotinic receptors can also be used to identify points when those quantities are known or can be estimated.

Landmark identification can also be aided by operations specific to the target. For example, the ON and OFF landmarks may be defined, by way of example, as points occurring when CMAP amplitude reaches 1-2% and 98-99% of Cmax respectively. In one implementation, when data collection frequency or intrinsic variability of data makes clear identification of this proportion difficult, an adjacent segment of the recovery curve where slopes are more easily determined is identified and then the segment to ON or OFF is extrapolated to the targeted proportion. In one implementation, this extrapolation is via an average curve for the missing segment. In another implementation, pharmacokinetic/pharmacodynamic parameters are estimated (as described herein) to modify the average curve specific to the individual.

The EC25 (e.g., point 463 (or C1 EC25 as viewed in FIG. 4B), EC50 (e.g., point 465 (or C2 EC50) as viewed in FIG. 4B), and EC75 (e.g., point 467 (or C1 EC75 as viewed in FIG. 4B) landmarks are identified by amplitudes matching their respective proportions of Cmax with timing identified via the imputation or extrapolation methods described above. Additional steps that may further refine EC50 identification may include identification as the point where the maximal recovery curve slope occurs or identification of the point occurring at the midpoint of the timespan between RP and PP.

FIG. 5 illustrates a flowchart of a method 500 for measurement of NMB and prediction of unobserved recovery events according to an embodiment of the present invention. Method 500 may be performed during a surgical or other medical procedure to (manually or automatically) adjust administration (or dosage) of NMB or other agents. Analysis begins when the presence of a recovery pattern is first identified at operation 505 (from stimulation of muscles in substantially the same manner described above). This state is determined when a consistently positive slope of an ordinal CMAP recovery curve is identified. Data points obtained following identification of a recovery pattern accrue with time at operation 510.

As sufficient data accumulates, some or all landmarks (e.g., ON, OFF, RP, PP, EC25, EC50, EC75, etc.) are identified at operation 515 based on the available data and the processes described above.

In operation 520, Cmax is estimated. In a preferred embodiment, Cmax is calculated using an equation derived from the Hill fractional effect equation. The Hill fractional effect equation is given here as FE=[N]^{γ}/([N]^{γ+}[N₅₀])^{γ}, where FE is the fractional effect, [N] is the concentration of an antagonist ligand, [N₅₀] is the antagonist ligand concentration at EC50, and γ is the Hill coefficient. The following proof is shown for a specific scenario where the [N] represents the effective concentration of a nondepolarizing NMB agent and the agonist is the junctional ACh in effectively constant concentrations associated with each ordinal CMAP. In particular, C1 and C2 recovery curves are chosen as an example and in this scenario, the fractional effects of NMB are 1−C1% and 1−C2% respectively. The above FE equation can be rearranged to solve for [N₅₀] at two time points a and b and the following four equations are generated:

[N₅₀₋₁]^{γ}=C1%ₐ*[Nₐ]^{γ}/(1−C1%ₐ);

[N₅₀₋₁]^{γ}=C1%_{b}*[N_{b}]^{γ}/(1−C1%_{b});

[N₅₀₋₂]^{γ}=C2%ₐ*[Nₐ]^{γ}/(1−C2%ₐ);

[N₅₀₋₂]^{γ}=C2%_{b}*[N_{b}]^{γ}/(1−C2%_{b});

where [N₅₀₋₁] and [N₅₀₋₂] are the EC50 concentrations of NMB for C1 and C2 respectively, and where [Nₐ] and [N_{b}] are the NMB concentrations present at times a and b respectively. The distinct expressions for [N₅₀₋₁]^{γ} and [N₅₀₋₂]^{γ} are then set equal and those expressions are solved for [Nₐ]^{γ}/[N_{b}]^{γ} resulting in the following equations:

[Nₐ]^{γ}/[N_{b}]^{γ}=(1−C1%ₐ)*C1%_{b}/((1−C1%_{b})*C1%ₐ);

[Nₐ]^{γ}/[N_{b}]^{γ}=(1−C2%ₐ)*C2%_{b}/((1−C2%_{b})*C2%ₐ).

The expressions for [Nₐ]^{γ}/[N_{b}]^{γ} are then set equal to each other resulting in:

(1−C1%ₐ)*C1%_{b}/((1−C1%_{b})*C1%ₐ)=(1−C2%ₐ)*C2%_{b}/((1−C2%_{b})*C2%ₐ).

Recognizing that C1%=C1/Cmax and C2%=C2/Cmax and solving for Cmax the equation becomes:

Cmax=(C1ₐ*C1_{b}*C2_{b}+C1ₐ*C2ₐ*C2_{b}−C1_{b}*C2ₐ*C2_{b}−C1ₐ*C1_{b}*C2ₐ)/(C1ₐ*C2_{b}−C1_{b}*C2ₐ).

While C1 and C2 are used in the above example, when any two ordinal CMAP amplitudes Cₓ and C_{y} (where x and y are the ordinal numbers) are known at time points a and b, Cmax is derived using the equation Cmax=(Cₓ₋ₐ*C_{x-b}*C_{y-b}+Cₓ₋ₐ*C_{y-a}*C_{y-b}−C_{x-b}*C_{y-a}*C_{y-b}−Cₓ₋ₐ*C_{x-b}*C_{y-a})/(Cₓ₋ₐ*C_{y-b}−C_{x-b}*C_{y-a}). More generally, in another implementation, when four of the five values determined by Cmax and the four CMAP quantities at time points a and b across two ordinal recovery curves are known or estimated, the fifth quantity is derived. Furthermore, this equation is generalizable to use in any agonist/antagonist system where the agonist is present in two or more distinct and fixed concentrations, the different concentrations of agonist will produce the same maximal effect upon full elimination of the antagonist, and decay of the antagonist occurs in a manner that adheres to the Hill fractional effect equation

In another embodiment, calculation of Cmax is performed across a range of available data points. In one implementation, any two available recovery curves are utilized for Cmax calculation. In another implementation, up to all permutations of pairs of curves available are utilized to generate additional data, improve estimates, and reduce noise. In one implementation, results from a multitude of calculations are parsed using measures of central tendency, exclusion of extremes of results, percentile calculations, and/or averaging to limit aberrant values due to variability in data collection and as one manner to target data adhering to the Hill equation. In another implementation, NMB recovery data that conforms to the Hill equation is first identified and then the Cmax estimate is calculated or re-calculated from this subset of the data. Such data targeting may be achieved via a number of techniques including morphology assessment of ordinal CMAP recovery curves or linearity assessment of the data plots that will be linear when adherence to the Hill equation is present (e.g., logit(Cx) vs. time).

Additional techniques may be implemented when direct Cmax calculation is not possible or to confirm/refine the calculated Cmax value. In one implementation, Cmax can be estimated from the value observed when full recovery is achieved at some point in a surgical procedure (e.g., data obtained prior to initial NMB administration). In another implementation, Cmax may be estimated from the expected Cx/Cmax associated with an identified stage of recovery (as described herein). In another implementation, Cmax estimation is refined by finding the Cmax value that optimizes parameters from linearity measures. It is noted that in most instances, a Cmax estimate is utilized to produce linearity measures. However, prior Cmax estimates are refined by finding Cmax values that optimize parameters for the linearity measures (as described herein). In another embodiment, the Cmax estimate and its refinement are determined by a weighted average of some or all the above determination techniques.

Following Cmax estimation, the level of NMB is measured at operation 525. In one embodiment, the preferred measures are the ratio of the ordinal CMAPs to Cmax. For example, the recovery of the first CMAP is described as C1/Cmax and is labeled here as C1%. Similarly, C2/Cmax, C3/Cmax, and C4/Cmax are labeled as C2%, C3%, and C4% respectively with Cx % referring to any or all of these measures. C1% is similar to, but distinct from, the currently used T1% described above. The T1% measure is a ratio of the first response of the TOF to a reference value meant to reflect the muscle's response in the absence of NMB effects. The distinction from C1% comes from the fact that the T1%'s reference value is a single, unchanging value typically taken at the beginning of a procedure before any NMB is administered while Cmax is determined during the medical procedure and can be updated over time. Both T1% and C1% are direct measures of the suppressive effect of NMB with the T1% routinely underestimating the degree of recovery over time due to the T1% denominator's known tendency to exceed CMAP amplitudes measured after full recovery. In another embodiment, Cx % values are combined to give a mixed or overall picture of the recovery progress. In one implementation, the average of C1% and C4% is used to describe an overall progress toward recovery. This average has an advantage over other direct measures of NMB in current clinical use in that it is 0% prior to emergence of recordable CMAP responses, 100% as recovery of all CMAPs is completed, and produces a flatter sigmoid response vs. time that approaches linear for much of its course thus yielding a more evenly paced recovery progress with respect to time.

Measurement of the degree of NMB is central to performing clinical decision making at operation 527. Measurement determines how neuromonitoring tests will be performed and interpreted and guides decisions related to the patient's clinical status including need for NMB redosing, need for NMB reversal, application of correct interpretative criteria in neuromonitoring, and determining risk of extubation. Accordingly, measurement of NMB may determine actions for medical personnel to adjust administration (or dosage) of NMB or other agents. Further, measurement of NMB may determine actions at CPU 135 where it controls or provides information (or controls) to other medical devices, such as a drug infusion device to control administration of NMB according to the effect or dosage, control reversal agents according to the effect or dosage, etc. or control nerve stimulation and other monitoring aspects for a neuromonitoring system. Furthermore, an ability to measure the degree of NMB is permissive to the subsequent operations for recovery map creation (e.g., a recovery map includes contiguous recovery curves generated from predicted values). Improvement in measurement over current techniques directly improves the clinical and predictive operations noted.

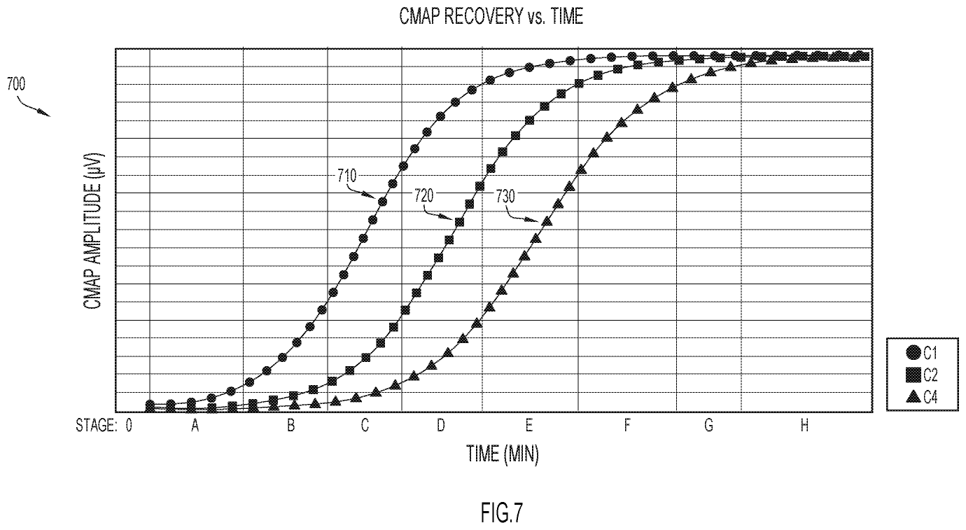

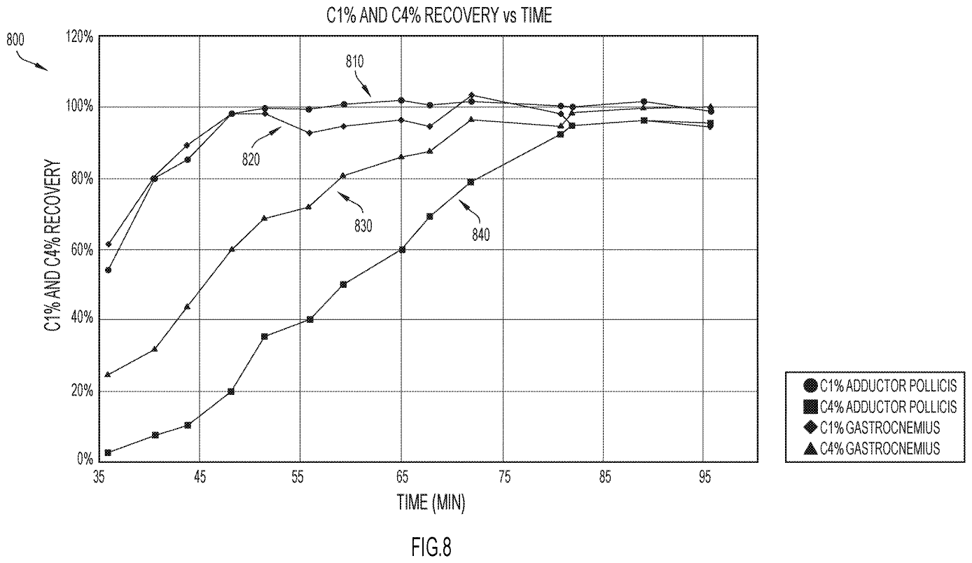

In an embodiment of the present invention, a broader stage of recovery is used as a NMB measurement device. A stage of recovery is identified by examining the relative recovery seen among the ordinal CMAPs vs. time. An example of one embodiment is shown in graphical representation 700 of FIG. 7 of CMAP amplitude (Y-axis) over time (X-axis). Circles, squares, and triangles represent idealized data points for C1, C2, and C4 respectively. Stages (A-H as viewed in FIG. 7) are drawn (along the X-axis) from patterns of response in curves 710 (for C1), 720 (for C2), and 730 (for C4). For this example, stage borders are set at each Recovery Point (RP), then each Plateau Point (PP), and finally at C4%=95%. Population data is used to correlate each identified stage to its expected degree of recovery and these results are applied as an early measurement of NMB prior to calculation of Cmax or as a simple visual assessment of recovery when graphical data are available. In an embodiment, a similar process of correlating the expected degree of recovery for slope-derived landmarks (e.g., RP and PP) is used to augment the estimates of recovery. In an embodiment, the stage of recovery is used when visual inspection of graphical data is desired without extensive numerical processing. In an embodiment, a number of different criteria for specifying the borders of each stage may be chosen.

Referring back to FIG. 5, data accumulates at operations 515 and 520 until the first recovery maps can be constructed at operation 530. Generation or update of “recovery maps” refers to the use of data observed during the recovery process to predict portions of the recovery curves that are not directly observed. Prediction of recovery with these maps is achieved through a variety of processes. In one embodiment, estimation of pharmacological parameters determining the recovery process is used to produce a map. In another embodiment, landmarks are used to create a map. In another embodiment, linearity measures otherwise used to indicate adherence to the Hill equation are in turn used to create a recovery map. In one embodiment, recovery maps are used to predict ordinal CMAP status and interrelations between ordinal CMAPs at any time of interest. In another embodiment, recovery maps are used to predict time of any specified ordinal CMAP status or interrelation between ordinal CMAPs. In another embodiment, recovery maps are used to determine the dosage of NMB agent that results in recovery events at a targeted time.

In another embodiment, pharmacokinetic/pharmacodynamic (PK/PD) modeling is used to construct a recovery map. While the PK/PD behavior of NMB agents is complex, the target period for this modeling is after there has been initial equilibration of NMB concentrations and simpler, first order kinetics can be applied. An estimate of the effective biophase concentration of the NMB agent (hereafter denoted as [N]) is given by the first order kinetics equation [N]=[N₀]e^{-kt} where [N₀] is an initial concentration of NMB, t is the measured time after the [N₀] level is reached, and k is the rate constant. [N₀] can be first estimated by adapting the NMB agent's degree of ACh receptor occupancy (θ) at a point on the recovery curves where this quantity has been determined. In another implementation, the NMB effective concentration is related to points of known NMB receptor occupancy by the Hill-Langmuir equation for non-cooperative systems [N]=θ*Kd/(1−θ), where Kd is the dissociation constant for the NMB agent. Reference values for the rate constant, k (or its equivalent, half-life) vary widely for NMB agents and are dependent on age, hepatic function status, and even the techniques used to determine it. The first order kinetics equation can be solved for k if [N] is defined at two points. As a result, in one embodiment, a patient-specific k value is calculated using [N] derived from at least two θ values associated with identified landmarks. Once an [N₀] is established and a patient-specific k is determined, the estimated [N] at any time point is calculated. In an embodiment, expected θ values for many points of interest on the ordinal CMAP recovery curves are determined using pooled [N] results across multiple subjects thus refining reference values and establishing new reference θ values at other points including any value of C1%. In another embodiment, θ values are drawn from known reference values.

Of note, the “effective” biophase concentration is meant to incorporate variable influences of anesthetics, systemic variables, and medications on the action of NMB. These influences may be altered during a procedure and, thus in a preferred implementation, pharmacological parameters (including [N]) are iteratively recalculated throughout a given procedure. In another implementation, known variables are tracked with expectations for [N] altered based on compensatory adjustments specific to the variable altered.

In another embodiment, the PK/PD recovery map is created by solving the Hill fractional effect (FE) equation: FE=[Nₜ]^{γ}/([Nₜ]^{γ}+[N₅₀]^{γ}) for all relevant times (t) where [Nₜ] is the NMB effective concentration at time t, [N₅₀] is the effective concentration at EC50, γ is the Hill exponent, and FE is 1−Cₜ/Cmax where Cₜ is the ordinal CMAP amplitude of interest at time t.

In an embodiment, the Hill exponent (γ) is estimated for each of the ordinal CMAP recovery curves of interest. In one example implementation, two points on a single recovery curve where the fractional effect has been calculated/estimated at times a and b are utilized. Then solving the fractional effect equation for [N₅₀]^{γ} at both points and then setting the associated values for [N₅₀]^{γ} equal to each other allows for calculation of the associated Hill exponent as follows: γ=ln{Cx %_{a*}(1−Cx %_{b})/(1−Cx %ₐ)*Cx %_{b}}/ln{[N_{b}]/[Nₐ]}. In another example implementation, when [N] and the fractional effect have been determined/estimated at a single point and the associated [N₅₀] is known/estimated, the equation γ=ln(Cₓ/(Cmax−Cₓ))/ln([N₅₀]/[N]) may be used. In another embodiment, the Hill exponent is estimated as the absolute value of the slope of the line resulting from a plot of logit(Cx %) vs ln[N]. In another embodiment, the Hill exponent is estimated from the maximum slope of Cx % vs. [N]. To this purpose, the derivative of the Hill fractional effect equation with respect to [N] (dCx %/d[N]=−γ*[N50]γ*[N]^{(γ-1)}/([N]^{γ}+[N₅₀]^{γ})²) is applied for [N]=[N₅₀] yielding maximum slope z=−γ/(4*[N50]) in units of percentage change per unit of concentration and rearranging, γ=z*(−4)*[N₅₀]. In another embodiment, the estimated Hill exponent is refined by determining the value that yields a best-fit of the observed data to a modeled estimate. In one best-fit example implementation, the γ that minimizes squared residuals between the Hill fractional effect equation and observed CMAP amplitudes is used. In another example best-fit implementation, the γ that maximizes linearity measures for the logit(Cx %) vs. time plot is used. In a preferred embodiment, the Hill exponent is determined for each recovery curve. In another implementation, an estimate for one recovery curve is used as a first estimate of the Hill exponent for one or more other curves. In another implementation, a Hill exponent determined for one recovery curve is applied to one or more other recovery curves with a correction factor added based on typical expectations for differences across CMAP curves based on population data.

Once estimates of the Hill exponents are achieved, all elements of the fractional effect equation are available and a CMAP amplitude at time t (Cₜ) is given by the equation: Cₜ=Cmax(1−[Nₜ]^{γ}/([Nₜ]^{γ+[N}₅₀]^{γ})) where [N₅₀] is the EC50 concentration associated with the ordinal recovery curve of interest. Application of this equation for all relevant values of t and for each of the ordinal recovery curves results in a full PK/PD recovery map.

In one embodiment, a landmark-based recovery map is constructed. For this purpose, landmarks are first estimated from initially available data as described above. Once the most readily estimated landmarks are identified across each recovery curve of interest, landmarks from one recovery curve are estimated on other recovery curves where the landmarks have not been identified using the same amplitude. The time separation across the different recovery curves is indicated by other landmarks of the same designation (e.g., RP) that are already common to each curve. In one implementation, the resulting groups of landmarks are graphically displayed and used as a rudimentary version of a recovery map for visual evaluation and gross mental estimates of future recovery events. In another implementation, recovery curve segments among the landmarks are completed. Within the rapid recovery phase (e.g., the EC25-EC75 interval), linear extrapolation is used to identify yet-unidentified landmarks and to complete curve segments within this phase. Segments of curves in the recovery or the plateau phases where curvature is greater are estimated as needed using the Hill fractional effect formula demonstrated above, Cₜ=Cmax(1−[Nₜ]^{γ}/([Nₜ]^{γ}+[N₅₀]^{γ})). For this implementation, EC50 and at least one other landmark with defined expectations for NMB receptor occupancy (and therefore their respective [N] values) must be identified and Cmax must be estimable. Nₜ is determined by applying [N]=[N₀]e^{-kt} where [N₀] is from one of the defined landmarks, t is the time from that landmark, and k is either an average value for the patient's age and hepatic function or is estimated from the two or more known landmarks using the technique described above for the PK/PD map construction. The remaining parameter, γ, may be an average value, may be an average value adjusted for the observed maximal slope of the curve, or may be estimated from the two or more known landmarks using the technique described above for the PK/PD map construction. Once selected curve segments are so estimated for one recovery curve, they may also be applied to the other recovery curves of interest using the observed time separation between the recovery curves.

Embodiments of the present invention recognize that each of the ordinal CMAP curves does not have the exact shape of those preceding or following it. In one implementation, expected variation in steepness (γ) and inter-curve time differences is incorporated according to the average differences across a population. In one embodiment, the landmark-based recovery map is targeted to periods where data is limited and a full PK/PD analysis is not considered possible or accurate, thereby allowing early analysis during recovery or an early map when redosing of NMB agent allows only limited data for analysis.

In addition, construction of the landmark-based recovery map, as well as the other recovery map types, may be augmented by artificial intelligence/machine learning estimations. Any quantity of any conventional or other machine learning models may be used (e.g., mathematical/statistical models, classifiers, feed-forward (fully or partially connected), recurrent (RNN), convolutional (CNN), or other neural networks, deep learning models, long short-term memory (LSTM), attention-based methods/transformers, large language model (LLM), etc.) that are configured/trained to estimate curve elements between landmarks. For example, the machine learning may be performed using a training set of various inputs (e.g., curve features, etc.) and corresponding desired outputs (e.g., estimated curve elements, etc.), where the machine learning model (e.g., neural network, etc.) attempts to produce the provided output and uses an error from the output (e.g., difference between produced and known outputs) to adjust weight (and bias) values (e.g., via backpropagation or other training techniques) of the machine learning model.

In another embodiment, alternative derivations of the Hill fractional effect equation are used to create recovery maps. In one implementation, a “logit-based” recovery map is created wherein the function logit (Cx %) is defined as ln(Cx %/(1−Cx %)) with x as the ordinal CMAP number. When setting 1−Cx %=[N]^{γ}/([N₅₀]^{γ}+[N]^{γ}) and solving for ln[N] we see logit(Cx %) and ln[N] are linearly related. Similarly, since ln[N] and time are linearly related, it also follows that logit(Cx %) and time are linearly related. In one embodiment, logit(Cx %) is calculated from observed data and the estimated Cmax value and then plotted vs. time. Once NMB equilibration has occurred and NMB recovery adheres to the Hill equation, the plotted data takes on the form logit (Cx %)=mt+b, where t is the timing of Cx, m is the observed slope of the plot, and b is the observed y-intercept. Rearranging this equation to give Cx as a function of time leads to Cx=Cmax*e^{mt+b}/(e^{mt+b}+1). Thus, using the observed slope and y-intercept from the logit plot, a logit-based recovery map is generated for all times of interest. In another implementation, other generalized linear models are utilized (e.g., probit function).

In another embodiment, it is recognized that the equation Cmax=(Cₓ₋ₐ*C_{x-b}*C_{y-b}+Cₓ₋ₐ*C_{y-a}*C_{y-b}−C_{x-b}*C_{y-a}*C_{y-b}−Cₓ₋ₐ*C_{x-b}*C_{y-a})/(Cₓ₋ₐ*C_{y-b}−C_{x-b}*C_{y-a}) can be rearranged to show a linear relationship in a plot of Cx/Cy and Cx/Cmax where x and y denote the ordinal number at a coincident time and where x>y (e.g., a linear relationship exists between the train-of-2 ratio and C2%). Furthermore, this linear relationship is described by Cx/Cy=m*Cx/Cmax+(1−m), where m is the slope and (1−m) is the y-intercept. In one implementation, when m is determined from observed data, Cmax is estimated, and either Cx or Cy is known, the remaining value is determined. In another embodiment, when m is determined and both Cx and Cy are known, the estimation of Cmax is refined by finding the Cmax value that maximizes a linearity measure or minimizes least square residuals for the equation Cx/Cy=m*Cx/Cmax+(1−m) across a range of known Cx, Cy pairs. In another embodiment, measures of linearity are applied to the Cx/Cy vs. Cx/Cmax data in order to identify intervals during which adherence to the Hill equation is expected.

As part of the recovery map creation process, critical parameters undergo best fit adjustments to optimize the maps'fit to the observed data. In one embodiment, the PK/PD or the landmark recovery map is optimized by minimizing least squares differences from the observed data for a solution where one or more of the rate constant (k), Hill coefficient (γ), and timing for a chosen landmark are adjusted. In another implementation, additional parameters may be adjusted including Cmax, landmark occupancy (θ) values, the disassociation constant (Kd), or amplitudes for slope-derived landmarks (e.g., RP or PP). In another embodiment, the logit recovery map is optimized by maximizing measures of linearity for logit plot slope (m), y-intercept (b), or Cmax. In another implementation, the logit map is optimized by minimizing least square differences between the map and observed data for a solution adjusting one or more of the logit plot slope (m), logit plot y-intercept (b), or Cmax. In another embodiment, Cmax estimates are refined by maximizing linearity measures for derivations intended to show linearity (e.g., the Cx/Cy=m*Cx/Cmax+(1−m) linear relationship). In another embodiment, parameters from one recovery map are optimized to fit a different recovery pattern which is preferred at the time. In one implementation, best fit adjustments are made with use of commercially available software (e.g., Excel Solver, Matlab, etc.). In another embodiment, artificial intelligence or machine learning is used to optimize parameters that provide the best fit of the recovery maps to the observed data. In another implementation, the best-fit for mapped curves is determined from a combination of these techniques. Any quantity of any conventional or other machine learning models may be used (e.g., mathematical/statistical models, classifiers, feed-forward (fully or partially connected), recurrent (RNN), convolutional (CNN), or other neural networks, deep learning models, long short-term memory (LSTM), attention-based methods/transformers, large language model (LLM), etc.) that are configured/trained to optimize the parameters that provide the best fit of the recovery maps. For example, the machine learning may be performed using a training set of various inputs (e.g., parameter values, etc.) and corresponding desired outputs (e.g., optimized parameter values, etc.), where the machine learning model (e.g., neural network, etc.) attempts to produce the provided output and uses an error from the output (e.g., difference between produced and known outputs) to adjust weight (and bias) values (e.g., via backpropagation or other training techniques) of the machine learning model.

An aggregate recovery map that is determined to be the best predictor of recovery events is selected at operation 535. The selection of this map may include averaging of two or more available maps or may be comprised of only a single recovery map deemed to be the best predictor. The basis of selection for the aggregate map is a patient-specific process with selection criteria including the quality of fit to the observed data, the consistency with other maps, the quality of data used to create the individual maps, the quantity of data available to create the individual maps, and the portion of the recovery process from which the maps are derived. In one example implementation, the PK/PD recovery map is assigned a weighting of x % while the logit recovery map is assigned a weighting of 1−x % and then x % is determined by the value that minimizes least squared residuals from the observed data.

The aggregate recovery map predicts any future (or prior) recovery event up to the point of full recovery of C4 at operation 540 and provides dosing guidance (e.g., for enabling the muscle to attain recovery at a certain time). Medical personnel may adjust administration (or dosage) of NMB or other agents according to the dosing guidance. Further, CPU 135 may control or provide information (or controls) to other medical devices, such as a drug infusion device to control administration of NMB based on a determined dosage, reversal agents based on a determined dosage, etc. In one embodiment, these predictions identify the timing of a specific event. In another embodiment, these predictions predict the recovery state at a specified time. In one embodiment, such recovery state or recovery events include ratios among any of the ordinal CMAPs, ratios between any of the ordinal CMAPs and Cmax, amplitudes of CMAPs, slopes of recovery curves, or component calculations or values used in creation of the recovery map.

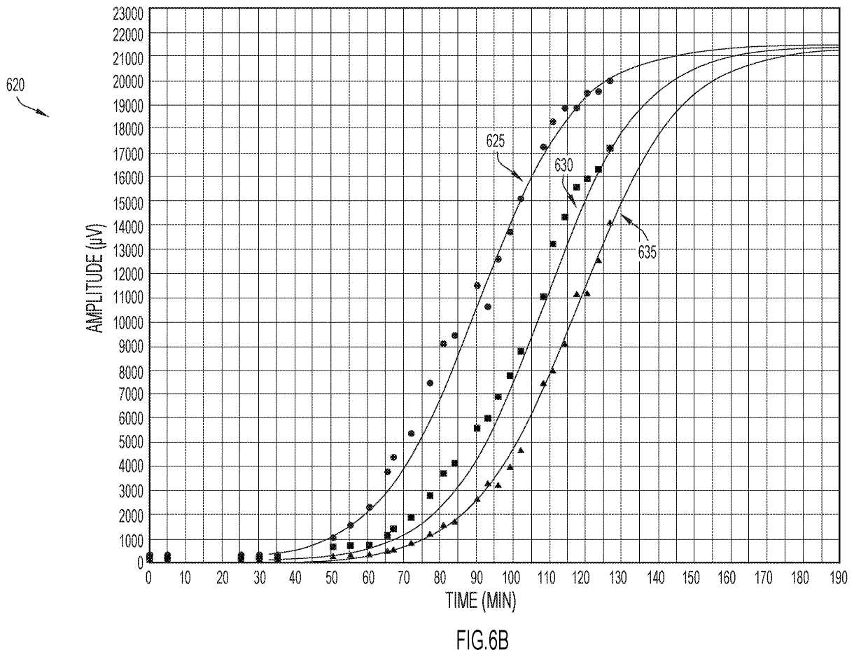

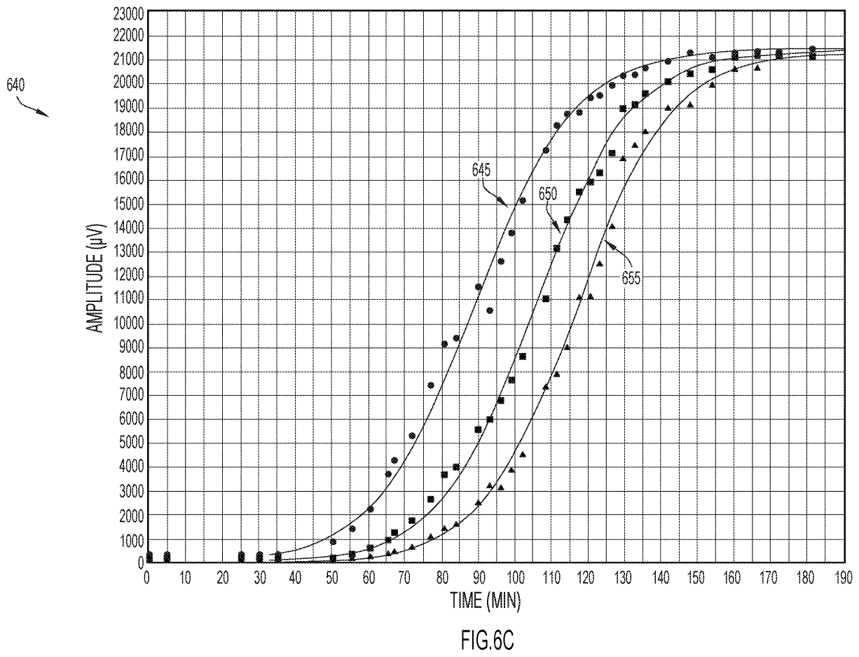

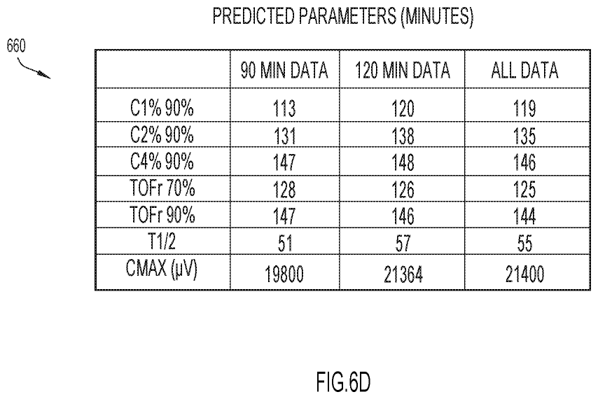

New data is examined to determine if their recovery pattern is consistent with the expected recovery path at operation 545. When no deviation in recovery is detected at operation 545, there is a return to operation 510 where the new data is added to the process in a continuous update of the analysis. As data is added, the aggregate recovery map is continually improved with progressively more precise prediction of yet-unrecorded recovery events. An example of this is seen in FIG. 6A-6D which illustrate the progressive update of a recovery map as data accumulates (from FIG. 6A to FIG. 6C) with data points for C1, C2, and C4 shown as circles, squares, and triangles respectively. FIG. 6A shows a graphical representation 600 of CMAP amplitude (Y-axis) over time (X-axis) for curves 605 (C1), 610 (C2), and 615 (C4). Graphical representation 600 demonstrates prediction of late recovery from an early point in the recovery process corresponding to a point where C1 (curve 605) and C2 (curve 610) reemergence is underway while C4 (curve 615), and therefore the TOFr, are yet to show significant recovery.

FIG. 6B shows a graphical representation 620 of CMAP amplitude (Y-axis) over time (X-axis) for curves 625 (C1), 630 (C2), and 635 (C4). Graphical representation 620 demonstrates prediction of recovery from a later point in the recovery process, but before Cmax is reached for any curve.

FIG. 6C shows a graphical representation 640 of CMAP amplitude (Y-axis) over time (X-axis) for curves 645 (C1), 650 (C2), and 655 (C4). Graphical representation 640 shows a complete data set with the final modeling of the recovery curves.

FIG. 6D shows a table 660 with prediction of parameters generated from the data sets of FIG. 6A-6C, where the prediction of events with the limited data of FIG. 6A varies from complete data set map's findings of FIG. 6C by at most six minutes despite prediction of events as far as fifty-six minutes prior.

Deviation from the ongoing recovery course may be identified in various manners at operation 545. In one embodiment, a deviation may be identified when data falls outside of the predicted path of recovery beyond that due to expected variability. In one implementation, the bounds of expected variability are a preset limit. In another implementation, the bounds of variability are based on the variability previously observed in the patient. In another embodiment, assessment of continued linearity in measures expected to be linear is utilized. In one implementation, deviation from linearity is suspected when linearity measures fail to remain within defined limits (e.g., a decrease in Pearson's R²) with loss of linearity confirmed when subsequent data obtained soon thereafter (e.g., within a 5-minute window) fails to show return to the previous pattern. In another embodiment, abrupt alteration of data beyond targeted limits is utilized. In another implementation, combinations of the above techniques applied across one or more ordinal CMAP recovery curves are utilized.

When disruptions of recovery are detected as determined at operation 545, actions are performed to identify the cause. A determination of whether an NMB reversal agent has been administered is performed at operation 550. A confirmation of administration of reversal may be indicated directly as a user's entry to the device or as an automatic signal from the anesthesiologist's electronic drug log. Alternatively, the detection of reversal can be made presumptively from the data and is indicated by a rapid rise in ordinal CMAP quantities occurring over a period of minutes beyond that appropriate to the NMB depth at the time.

When reversal agent administration is signaled or detected as determined at operation 550, a determination is performed of whether the clinically intended effect (e.g., TOFr>90% or its equivalent) is reached once the effect of the reversal agent has peaked. When the intended effect of reversal is not reached as determined at operation 555, the most recently determined aggregate recovery map is applied at operation 560 with the reversal causing movement forward on that map's time scale to the point where projected CMAP amplitudes match those detected after reversal equilibration. At that point, the map is then recalibrated to the current time with subsequent recovery events predicted in line with expectations of the later portion of the prior map. In another embodiment, the recalibrated recovery map is adjusted for the ongoing impact of the reversal agent on the decay of [N]. The output (or recalibrated recovery map) is applied to operation 510 where the accumulated data is incorporated into new recovery map versions, and is applied to operation 540 where recovery predictions and dosing guidance are provided based on the altered recovery map. In one embodiment, dosing guidance is adjusted for the further impact of the reversal agent on the effective concentration of NMB agent additional dosing at operation 540 based on the recalibrated recovery map from operation 560. Medical personnel may adjust administration (or dosage) of NMB or other agents based on the updated dosing guidance. Further, CPU 135 may control or provide information (or controls) to other medical devices, such as a drug infusion device to control administration of NMB based on a determined dosage, reversal agents based on a determined dosage, etc.

When clinically adequate reversal is detected as determined at operation 555, TOF measurement continues at operation 565 with ongoing assessment for the re-emergence of an effect of NMB (“recurarization”). In one embodiment, recurarization is detected by an identifiable downward trend in ordinal CMAP quantities outside the expected degree of variation. In another embodiment, recurarization is indicated by measurements consistently falling within any chosen criteria including amplitude change in one or more ordinal CMAPs, falling below a given Cx % level, or identification of an adverse change in measures based on ratios of ordinal CMAPs.

When recurarization is detected as determined at operation 565, the recurarization is determined to be intentional (due to administration of the NMB agent) or unintentional at operation 575. Intentional administration of NMB agent recurarization is indicated by the device user, or from an automatic signal from the anesthesiologist's electronic drug log. When the recurarization is intentional as determined at operation 575, the process repeats as described above from operation 505 and the analysis process restarts.

When the recurarization is unintentional (no intentional administration of NMB agent) as determined at operation 575, the device alarms at operation 580. In an embodiment of the present invention, identification of unintentional recurarization is made. Recurarization in this context refers to the delayed re-emergence of NMB following initially effective administration of a reversal agent due to waning over time of the reversal agent's effect while significant plasma levels of the NMB agent persist. Recurarization in this context is typically unexpected and, if it occurs after removal of ventilation and other support systems, it can be catastrophic. Recognition thereof by the current device signals a need for immediate supportive care which may include administration of additional reversal agent and ventilatory assistance. In one embodiment, the device controls a medication/infusion system allowing automatic reversal agent administration upon detection of unintentional recurarization.

In the absence of recurarization, assessment continues at operation 565 until the risk of recurarization has passed. In one implementation, the device user determines when the risk has passed and no further analysis is needed leading to the end of analysis at operation 570. In another implementation, the expected [N] is estimated based on the last available recovery map with adjustment, as appropriate, for alteration of NMB clearance due to the reversal agent. When the estimated [N] is sufficiently low that no significant NMB effect is expected in the absence of a reversal agent, the device indicates that the period of primary risk has passed. In one implementation, this state of low risk is signaled to the user who may then choose to end the analysis and, in another implementation, the device automatically terminates assessment when the estimated period of primary risk has passed.

An additional case exists when clinically complete recovery from NMB occurs (as determined at operation 550) due to normal physiologic elimination of NMB from the body. This case is identified when, in the absence of a reversal agent, amplitudes reach Cmax with average slopes reaching 0 for all recovery curves, and this process occurs in line with the preexisting recovery map. “Reversal” in this case is by definition full (as determined at operation 555) and it is expected that recurarization cannot occur (as determined at operation 565) with the device user ending analysis at operation 570 if no further NMB agent administration is anticipated.

When deviation from expected recovery occurs as determined at operation 545 and is not attributed to reversal as determined at operation 550, detection of an NMB agent administration is determined at operation 585. In this scenario, an abrupt decline in ordinal CMAP amplitudes occurs that is associated with intentional administration of an NMB agent. In one embodiment, NMB administration detection is based on the pattern of alteration in the ordinal CMAPs. In one implementation, detection is via external input from the device user or from the user's electronic medication log.

When NMB administration is detected as determined at operation 585, but the specific NMB agent administered is unknown, it is initially assumed to be the same agent previously employed. In this situation, and when the same agent is confirmed via user input, a new cycle of analysis begins and the above process repeats from operation 505. The previous analysis is retained and used as the best estimate of subsequent recovery as subsequent data refines the prior analysis.

Rarely, the NMB agent used at operation 585 is different from the one upon which previous analysis is based. In one embodiment, this change is indicated by external user input at operation 585 and includes identification of the new agent so that the subsequent PK/PD analysis can be correctly performed specific to the agent used. Operation 505 ensues with fresh analysis not informed by the prior recovery map. In another embodiment, a new NMB agent is utilized but no user input indication is made. Operation 505 ensues with new data producing a recovery pattern inconsistent with the prior recovery map detected at operation 545 and the recovery disruption assessment is ultimately performed at operation 590.

The recovery disruption assessment at operation 590 is activated when signal alterations detected at operation 545 do not fit a pattern suggesting NMB reversal or new NMB administration. These patterns are categorized at operation 590 as minor-limited or major with the identified category determining subsequent analysis upon returning to operation 505. Minor-limited signal alterations determined at operation 590 involve data that is altered for a relatively brief timespan of data points before the initial recovery pattern resumes in the prior pattern after a brief interval. In one embodiment, these changes are addressed by eliminating the epoch with the altered pattern from the analysis and adjusting the timeline so that the resumed recovery pattern occurs along the previously predicted path (in effect the epoch of change is “cut out” of the timeline and prior analysis resumes as if no alteration was made). Causes of minor-limited change are commonly undetermined but a prototypical cause may be a microdose of NMB which may occur when an IV line that previously held an NMB agent is flushed. Minor changes that do not return to a prior pattern of recovery are treated as “major” regardless of the degree of changes.

Major alterations show greater and relatively abrupt degree of change with possible causes including alteration of the signal acquisition system, administration of a different NMB agent, major alteration of the anesthetic regimen, or new onset nerve or muscle dysfunction. Analysis after major changes may incorporate prior elements of prior recovery maps according to cause. For example, a new NMB agent or a major anesthetic change is likely to require entirely new construction of recovery maps; dislodgment of electrodes may be fixable with most if not all elements of the prior recovery maps remaining applicable; and nerve dysfunction may be reversible (e.g., release pressure on the nerve) with prior maps remaining applicable. However, in many scenarios no clear cause will be identified and regardless of cause, analysis resumes at operation 505. Subsequent analysis will determine if the data suggests same or similar recovery map parameters (e.g., rate constant, Hill coefficient, slope and y-intercept of the logit plot, and Cmax) with these elements preserved as appropriate.

Gradual signal alterations are an expected component of longer medical procedures where multiple cycles of NMB administration may be encountered. In this scenario, there is commonly a more subtle deviation from a previously predicted recovery map (not identified at operation 545), thereby leading to the otherwise uneventful update of data in operation 510. Compensation for gradual effects instead occurs as part of usual data update and best fit adjustments of recovery maps. In one embodiment, results are compared across different recovery cycles (periods of recovery occurring between NMB dosing) with the current cycle compared to past ones to identify the presence of gradual change and to quantify the associated rate of change. In another embodiment, recent data is weighted more in analysis than older data. In one implementation, the weighting of new vs. old data is determined by the rate of change identified between recovery cycles. Examples of changes in the physiologic milieu that may produce minor-gradual effects include altered pharmacologic factors (e.g., anesthetic agents, steroid administration, select antibiotics, magnesium, etc.) or systemic factors (e.g., temperature, hepatic function, pH balance, etc.) that impact the expected recovery of NMB due to altered NMJ function or altered NMB elimination.

Bolus administration of NMB often occurs in repeated doses through a medical procedure in order to maintain a patient within a targeted range of muscle relaxation. The targeted range of muscle relaxation may change during the medical procedure and the degree of NMB must be adjusted to meet these goals. In one embodiment, the NMB recovery map directs planning and administration of additional NMB. In one implementation, additional dosing of NMB is guided when the recovery map predicts waning effects of NMB during a time when it is expected that NMB is still needed. In another implementation, improved measurement of NMB allows tight targeting within the therapeutic range. In another embodiment, the status of the NMB following a planned dose of NMB is predicted. To this end, the effective concentration following NMB administration is estimated to increase from the current [N] by an amount equal to the dose/Vd, where Vd is the patient's volume of distribution. The resulting estimate for [N] following NMB administration may be associated with an NMB status using the recovery maps. In another embodiment, the dosage needed to reach a specific post-dose, post-equilibration NMB measurement is determined. If, for example, a C1% of 20% is targeted, the then-current recovery map indicates the [N] associated with that degree of NMB and the associated dose recommendation is given by the (desired change in [N])*Vd.

In another embodiment, the dose of NMB is determined that results in a specific NMB recovery event (e.g., TOFr>90%) at a specified time. To this end, the recovery map yields an effective concentration associated with the targeted recovery event, [Nₑ]. Next, the post-dosing concentration ([N_{d}]) that will result in [Nₑ] occurring at the correct time is calculated via [N_{d}]=[Nₑ]/e^{-k*Δt} where k is the rate constant and Δt is the time between the current time and the time of the targeted recovery event. Finally, the targeted dose that will produce [N_{d}] when starting with the current effective concentration [N_{c}] is calculated via: dose=([N_{d}]−[N_{c}])*Vd.

In another embodiment, the volume of distribution (Vd) is estimated. In one implementation, Vd is estimated using known values for volume of distribution related to the NMB agent used which are then adjusted as appropriate for weight, age, gender, body fat, and associated disease. In another implementation, a patient-specific “effective Vd” is calculated as Vd=dose/([N_{d}]−[Nₚ]), where [N_{d}] is the effective concentration after dosing and [Nₚ] is the effective concentration prior to dosing. [Nₚ] is known when it is the initial dose (i.e., [Nₚ]=0) or it is estimated from a PK/PD recovery map that has been generated prior to dosing (i.e., it is the [N] associated with the C1% value prior to dosing). The [N_{d}] is estimated using [N_{d}]=[Nᵣ]/e^{-kt}, where k is the rate constant, and [Nᵣ] is an effective concentration predicted by the PK/PD recovery map t minutes after dose administration. It is noted that [N_{d}] is not meant to reflect a specific bodily concentration immediately after dosing, but rather, it is a hypothetical instantaneous value that would result in [Nᵣ] if the first order kinetics that will be present after equilibration were present from the time of dosing. Thus, this approximates the volume corresponding to the more relevant post-equilibration state.

In another embodiment, the recovery map guides the use and selection of NMB-reversal agents. When the recovery map indicates that NMB may not sufficiently wane prior to targeted procedural events, planning for use of a reversal agent ensues and the expected degree of NMB at the time when return of muscle function is needed also impacts the specific reversal agent chosen. In another embodiment, the predictive maps and tailored dosing of NMB allow avoidance of NMB-reversal agents which has a number of associated benefits. First, if NMB is allowed to recover without reversal, the reintroduction of NMB, should that be required, is greatly facilitated due to the high dosages of NMB required to overcome preceding reversal and due to the reduced predictability of clinical effect. Second, awareness of when a targeted level of NMB recovery occurs allows planning for actions that require a specified degree of muscle function (e.g., accurate interpretation of intraoperative neuromonitoring testing or extubation). Third, targeted dosing allows native recovery at a time of choosing for actions that require a specified degree of muscle function. Fourth, use of a reversal agent may be associated with recurarization where the reversal agent's effect wanes before the effects of longer-acting NMB agents. If this occurs, the NMB agent resumes its clinical effects which can be clinically hazardous if the NMB reactivation is not anticipated, especially when a patient is not maintained on ventilatory support. Finally, modern NMB reversal agents may be a significant monetary cost and use is more likely to be avoided when there is awareness of when recovery occurs without intervention or when targeted dosing is used to limit the need for reversal.

Medical personnel may adjust administration (or dosage) of NMB or other agents based on these determinations. Further, CPU 135 may control or provide information (or controls) to other medical devices, such as a drug infusion device to control administration of NMB based on a determined dosage, reversal agents based on a determined dosage, etc.

In an embodiment of the present invention, parameters determined in the process of measuring NMB, creation of recovery maps, dosing, and other aspects of the above techniques are preserved to aid in the measurement, recovery prediction, and dosing of NMB in future medical procedures. In one embodiment, measurement is aided in subsequent procedures via early estimation of Cmax based on prior Cx % findings previously associated with early landmarks. In another embodiment, recovery map creation is aided by preservation of a host of possible parameters translated from a previous medical procedure. These parameters may include using previously determined values for k and the Hill coefficients, using Cx % values to predict the associated [N], utilizing similar time differences between ordinal recovery curves across the spectrum of Cx %, utilizing similar times between landmarks on the same ordinal CMAP recovery curve, utilizing similar Cx % differences between different landmarks on the same ordinal CMAP recovery curve, utilizing similar relative slopes between differing recovery curves across the spectrum of Cx %, and utilizing prior values for slope and y-intercept for linearized measures (e.g., logit function). In another embodiment, initial dose determination in a subsequent medical procedure is guided by use of the observed depth of NMB resulting from the chosen initial dose or subsequent doses in the earlier medical procedure, the recovery maps generated in the earlier medical procedure, the volume of distribution previously calculated, and any other relevant data from the prior medical procedure. In an implementation, the dosing guidance is used to produce a desired depth of NMB. In another implementation, the dosing guidance is used to target a recovery event at a specific time. For all estimates and determinations resulting from translating parameters from one medical procedure, estimates are supplanted as patient-specific data for the new medical procedure is acquired and provides data specific to the milieu of that medical procedure for factors that impact either the action or clearance of NMB agents (e.g. anesthetic depth, anesthetic agent, body or limb temperature, etc.). In another embodiment, the TOF is performed in a different muscle than in the prior medical procedure with adjustment to the predictions based on known relative differences between the two muscles'recovery pattern.

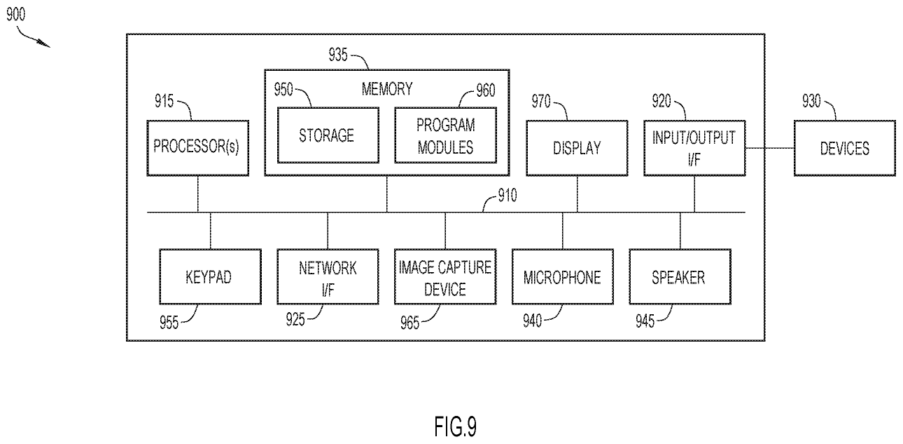

FIG. 8 illustrates how recovery patterns across different nerves and their target muscles may vary substantially according to an embodiment of the present invention. A graphical representation 800 of C1% and C4% recovery (Y-axis) over time (X-axis) includes curves 810, 820, 830, 840 for the adductor pollicis and gastrocnemius muscles in a single patient. In the example shown, curves 810 (C1%) and 840 (C4%) correspond to the adductor pollicis muscle, while curves 820 (C1%) and 830 (C4%) correspond to the gastrocnemius muscle. While the C1% for both muscles recovers similarly, the C4% for the adductor pollicis is delayed relative to the gastrocnemius. Thus, the adductor pollicis C4 recovery differs from the gastrocnemius C4 recovery, whereas the temporal course of C1 recovery for these muscles is similar. It is assumed that once NMB equilibrates, each neuromuscular junction encounters roughly the same local plasma concentration of NMB and, when differences exist, they occur in stereotyped relationships through the body. As a result, it is expected that differences in NMB recovery patterns across different muscles relate primarily to physiological differences at each muscle's neuromuscular junction (e.g., muscle fiber type mix or decremental ACh release) and that the sum of NMB concentration and neuromuscular junction physiological differences result in recovery patterns that differ in stereotyped ways for each of the ordinal CMAPs. Thus, in one embodiment, the differences in muscle recovery patterns are characterized to determine equivalent responses at any given degree of NMB depth. These differences are characterized across a population of subjects using each ordinal CMAP's fractional effect (Cx %) or combinations thereof resulting in determination of equivalent effects across the NMB spectrum. Next, all nerve-muscle pairs that are of interest for TOF testing are so-characterized with findings for the muscles characterized as proportions of a single reference nerve-muscle pair (e.g., ulnar-adductor pollicis). On average, recovery differences between muscles are expected to be transitive, meaning that if equivalent responses between the reference muscle and two distinct muscles are known, then equivalent responses for the two non-reference muscles can be estimated. Thusly, the “systemic level of NMB” is then described by any given TOF test's results converted to the reference muscle equivalent. In some situations, it may also be useful to describe other NMB values/events in terms of equivalence to the reference muscle and these may include relative timing of landmarks, steepness of the recovery curves (Hill coefficient), time separation in ordinal recovery curves, proportionate amplitude separation between landmarks, or parameters describing the logit function results.

It is noted that currently utilized measures of T1% or TOFr are inadequate to characterize the systemic level of NMB because T1% only assesses one recovery curve when the other ordinal curves may vary significantly between muscles (as demonstrated in FIG. 8). The TOFr shows an even greater loss of granularity of assessment due to use of a ratio of two independently relevant values that only provides any indication of NMB depth late in the recovery process.

Despite the absence of a previous description of a systemic level of NMB framework, the principles of the transitive properties across muscles are utilized routinely in clinical practice. This is because the muscle tested with the TOF is commonly not the muscle whose function is of primary interest and even when it is, it is rarely the only muscle of interest. For example, a TOF in the hand may be tested in order to determine if the diaphragm and intercostal muscles have sufficient function to allow respiration without external ventilatory assistance. Here, the TOF results must predict the degree of NMB suppression of muscle function in muscles that are not easily accessible to TOF testing. In general, to make these predictions, muscle function must first be associated with NMB measures using a population of subjects across the relevant spectrum of NMB depth. The quality of the resulting predictions depends on the intrinsic variability of the function relative to NMB depth, the accuracy of the NMB measure used to predict the underlying NMB depth, and the quality of the data used to characterize the measure/function relationship. In one embodiment, muscle-specific findings of clinical function are mapped to the systemic level of NMB using Cx % measures (e.g., obtained via device 100 in substantially the same manner described above).

As another example, intraoperative neuromonitoring assessment of motor evoked potentials may evaluate a dozen simultaneous muscles of interest while the muscle used for TOF testing may be just one (or none) of those 12 muscles. In the presence of partial NMB, the TOF test in this case is intended to serve as a manner to predict the impact of that NMB on the other muscles. In order to make quantitative predictions, the TOF test used must be mapped to the test results for every muscle of interest across the full spectrum of partial NMB. In current usage, such muscle-specific predictions are poorly quantified and this relates to both imperfections of the TOF count, TOFr, and T1% as measures of NMB depth as well as incomplete mapping of the TOF findings to the expected results of the functional test. With better measures and further characterization, a clearer quantitative understanding may be established whereby the proportionate impact of NMB on each muscle can be predicted based on the NMB measure's indication of the NMB depth. In one embodiment, clinical tests generating muscle responses are mapped to the systemic level of NMB.

Once such proportionate effects on muscle tests are determined, the suppressive effect of NMB can be removed from the test result. To this end, the measured functional test quantity is multiplied by the reciprocal of the proportionate residual muscle response predicted by NMB measure thereby yielding the expected result in the absence of NMB (e.g., if a muscle response is expected to be suppressed to 25% of its full response at a given level of NMB, then a clinical response quantified under those conditions is multiplied by four to remove the NMB effect). Once the effect of NMB is removed, results can more easily be compared to a baseline set of data obtained in the absence of NMB (or to a baseline set of data obtained in the presence of NMB that has been similarly normalized to the NMB-free state). In another embodiment, tests generating muscle responses in the presence of NMB are converted to the estimated equivalent result with the NMB effect removed.

Also of interest with functional tests is the likelihood that discrete changes of function or discrete changes in tests with muscle responses may occur (e.g., probability of need for reinstitution of ventilatory support or probability that a muscle quantity will fall to zero). In another embodiment, the probability of a specified degree of loss of clinical function is characterized using the reference muscle's Cx % measure across the NMB spectrum. In another embodiment, the probability of a specified degree of loss of muscle tested quantity is characterized using the reference muscle's Cx % measure across the NMB spectrum. In another embodiment, predictions of the impact of NMB on functional tests are further stratified by the initial signal quality which may include absolute quantities/amplitudes in the absence of NMB, preceding variability, and signal-to-noise ratio.

The construction of a map of the systemic level of NMB impacts clinical practice in a number of ways. First, it yields similar predictions of NMB's impact on clinical function regardless of the nerve-muscle pair used to perform a repetitive nerve stimulation test. This provides flexibility of assessment and supplants the common clinical practice of applying the same interpretative criteria to NMB measures regardless of the muscle from which it is obtained. The map of systemic NMB also incorporates the impact of NMB on functional tests of muscles not directly assessed by repetitive nerve stimulation. For intraoperative neuromonitoring, this aids in identifying new pathological loss of motor function (the primary goal of monitoring) by compensating for the effect of NMB and allowing more precise assessment as to underlying neurologic function. When neuromonitoring detects new neurologic dysfunction, a number of operations may be prompted including reversal of recent surgical actions, removal of surgical hardware, further decompression/resection, increase in blood pressure, or termination of the procedure. “Removal” of the unwanted impact of NMB improves the accuracy with which new neurological dysfunction is identified leading to more prompt and more appropriate further treatment of the patient when dysfunction is present with attendant rescue of at-risk neurologic function. This also allows the avoidance of unnecessary and potentially detrimental steps when the NMB would otherwise prompt a false positive alert. Removing this clouding effect of NMB improves the neuromonitoring both in improving the ability to identify new dysfunction and in the ability to avoid false identification of signal loss when no new dysfunction exists (false positive findings), thereby improving patient outcomes, reducing OR and hospital stays, and reducing the overall cost of care.

The systemic NMB map is predicated on use of direct measures of the suppressive effect of NMB and these are applied individually to the ordinal CMAPs across the spectrum of NMB. Muscular responses or function of interest vary in terms of which individual ordinal CMAP recovery is most relevant so NMB measure specific to each ordinal CMAP is important. A C1% response is most relevant to measures that involve a single stimulus as is the case with most motor evoked potentials. A C4% approximates maximal fade (e.g., the amount of fade stabilizes at approximately the C4's level if repetitive nerve stimulation is extended) and, therefore, best represents measures that follow tonic activation of the neuromuscular junctions. Such tonic activation is expected to occur with voluntary muscle function (including respiratory function) or trains of EMG activity during intraoperative neuromonitoring. A number of tests in neuromonitoring may vary in their Cx % relevance. For example, a motor evoked potential is often elicited with train pulses in quick succession. If, for example, two stimulus trains are so-delivered, this would activate the neuromuscular junction twice making C2 relevant to that instance. Intraoperative motor nerve conduction studies (“triggered EMG”) used to determine the threshold level of stimulation may vary in their Cx % relevance. When no NMB is present, testing is typically performed with repetitive stimulation above and below the threshold level until a precise level is determined with the Cx % irrelevant due to the absence of NMB. If this same testing is done in the presence of NMB, then the C4 is most relevant and the maximal fade in signals increases the likelihood of a falsely elevated threshold result. Alternatively, single stimuli at the threshold level used in clinical judgment makes the C1 most relevant and minimizes the likelihood of altered threshold findings related to the NMB.

As noted, the C4% is the measure most relevant to respiratory function. The determination of TOFr>90% is currently recommended as an indicator of sufficient respiratory muscle function to allow removal of ventilatory support. However, given that the TOFr is an indirect measure of NMB and given that this criterion was set as a manner to predict the direct measure of the suppressive effect of NMB, the reinstitution of a direct measure of NMB may improve accuracy by removing variability related to the TOFr's estimation of the direct measure. If less error is introduced, clinical criteria for judgment can be set more precisely avoiding longer recovery periods, thereby reducing operating room time and avoiding unnecessary use of NMB reversal agents. In another embodiment, the C4% is used for determination of return of respiratory function sufficient to allow removal of ventilatory support. Additionally, the average of C1% and C4% has attractive features for the same role. This measure is likely to yield similar or identical results since the C4% in the relevant range as the C1% is expected to have reached 100% when the C4% is in the 90% range. The advantage of the average is that it provides a much more gradual recovery pattern that is nearly linear through the majority of the NMB depth spectrum and, thus, provides a convenient measure to follow for progress toward recovery. In another embodiment, the average of C1% and C4% is used for determination of return of respiratory function sufficient to allow removal of ventilatory support.

In another embodiment, NMB assessment techniques that assess the movement of the muscle or target joint (“mechanical measures”), such as mechanomyography and acceleromyography, are also used to describe a systemic level of NMB with reference muscle using a mechanical measure. In another embodiment, mechanical measures are incorporated into an EMG-determined system of systemic level of NMB with an EMG response and used for the reference muscle. It is noted that the mechanical measures represent fundamentally different measures of NMB even when determined in the same muscle as the EMG measure and must first be independently assessed for equivalent values to the EMG reference muscle across the NMB spectrum.

In another embodiment, mechanical measures are used to produce the Cmax predictions and recovery maps described above. It is noted, however, that pharmacologically, the mechanical measures are further downstream in the physiologic sequence from neural depolarization to recording of a muscle response. As a result, there is greater likelihood of introduced skew in results with the differences from EMG responses indicating that this skew is substantial. With introduced skew, the correlation of muscle responses to occupancy may differ from EMG and adherence to the Hill equation may suffer, thereby impairing the accuracy of measurement and predictions. In one embodiment, introduced skew is directly corrected. In another embodiment, the introduced skew is corrected via usage of equivalent values in the reference EMG muscle used for determining the system level of NMB.

An example of a computing device 900 (e.g., implementing CPU 135 or other computing devices, etc.) is illustrated in FIG. 9. The example computing device may perform the functions of present invention embodiments described herein. Computing device 900 may be implemented by any personal or other type of computer or processing system (e.g., desktop, laptop, hand-held device, smartphone or other mobile device, etc.), and may be used for any computing environments (e.g., cloud computing, client-server, network computing, mainframe, stand-alone systems, etc.).

Computing device 900 may include one or more processors 915 (e.g., microprocessor, controller, central processing unit (CPU), etc.), network interface 925, memory 935, a bus 910, and an Input/Output interface 920. Bus 910 couples these components for communication, and may be of any type of bus structure, including a memory bus or memory controller, a peripheral bus, and a processor or local bus using any of a variety of conventional or other bus architectures. Memory 935 is coupled to bus 910 and typically includes computer readable media including volatile media (e.g., random access memory (RAM), cache memory, etc.), non-volatile media, removable media, and/or non-removable media. For example, memory 935 may include storage 950 containing nonremovable, non-volatile magnetic or other media (e.g., a hard drive, etc.). The computing device may further include a magnetic disk drive and/or an optical disk drive (not shown) (e.g., CD-ROM, DVD-ROM or other optical media, etc.) connected to bus 910 via one or more data interfaces.

Moreover, memory 935 includes a set of program modules 960 that are configured to perform functions of present invention embodiments described herein. The memory may further include an operating system, at least one application and/or other modules, and corresponding data. These may provide an implementation of a networking environment.

Input/Output interface 920 is coupled to bus 910 and communicates with one or more peripheral or external devices 930 (e.g., a keyboard, mouse or other pointing device, a display, sensing devices, etc.), at least one device that enables a user to interact with computing device 900, and/or any device (e.g., network card, modem, etc.) that enables computing device 900 to communicate with one or more other computing devices. Computing device 900 may communicate with one or more networks (e.g., a local area network (LAN), a wide area network (WAN), a public network (e.g., the Internet), etc.) via network interface 925 coupled to bus 910.

Computing device 900 may further include, or be coupled to, a touch screen or other display 970, a camera or other image capture device 965, a microphone or other sound sensing device 940, a speaker 945 to convey sound, and/or a keypad or keyboard 955 to enter information (e.g., alphanumeric information, etc.). These items may be coupled to bus 910 or Input/Output interface 920 to transfer data with other elements of computing device 900.

It will be appreciated that the embodiments described above and illustrated in the drawings represent only a few of the many ways of implementing embodiments for time-based methods and systems for the assessment and prediction of recovery from pharmacologic neuromuscular blocking agents. In addition, characteristics or features of embodiments of the present invention may be combined in any fashion to provide additional embodiments of the present invention.

A computing environment of the present invention embodiments may include any number of computer or other processing systems (e.g., client or end-user systems, server systems, etc.) and databases or other repositories arranged in any desired fashion, where the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system (e.g., desktop, laptop, hand-held devices, smartphones or other mobile devices, etc.), and may include any commercially available operating system and any combination of commercially available and custom software (e.g., communications software; server software; software of present invention embodiments; etc.). These systems may include any types of monitors and input devices (e.g., keyboard, mouse, voice recognition, etc.) to enter and/or view information.

It is to be understood that the software of the present invention embodiments (e.g., program modules 960, etc.) may be implemented in any desired computer language and could be developed by one of ordinary skill in the computer arts based on the functional descriptions contained in the specification and flowcharts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flowcharts may be modified in any manner that accomplishes the functions described herein. In addition, the functions in the flowcharts or description may be performed in any order that accomplishes a desired operation.

The software of the present invention embodiments (e.g., program modules 960, etc.) may be available or encoded on a non-transitory computer useable or readable medium (e.g., magnetic or optical mediums, magneto-optic mediums, CD-ROM, DVD, memory devices, etc.) of a stationary or portable computer program product, apparatus, or device for use with stand-alone systems or systems connected by a network or other communications medium. The computer useable or readable medium (or media) may include instructions executable by one or more processors to perform functions of present invention embodiments described herein.

A communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information. The database system may be implemented by any number of any conventional or other databases, data stores or storage structures to store information. The database system may be included within or coupled to the server, client, and/or other systems. The database systems and/or storage structures may be remote from or local to the computer or other processing systems, and may store any desired data.

The present invention embodiments may employ any number of any type of user interface (e.g., Graphical User Interface (GUI), command-line, prompt, etc.) for obtaining or providing information (e.g., measurements, landmarks, one or more recovery curves, data points, visualizations, NMB or other agents to administer, dosages, etc.), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

Aspects of the embodiments may include a method to estimate a degree of muscle recovery from non-depolarizing neuromuscular blocking agents comprising: applying a repetitive nerve stimulation to a motor nerve; quantifying each of the muscle responses resulting from repetitive stimulation; tracking the recovery pattern of each ordinal muscle response versus time; predicting the quantified muscle response after full recovery from NMB; identifying targeted portions of recovery curves measuring the degree of NMB effect; identifying when recovery from NMB adheres to specific pharmacologic patterns; and predicting all unobserved portions of recovery curves for each ordinal muscle response. An aspect may further include a device that performs the method described herein.

An aspect of the method excludes NMB assessment within a specified time of prior nerve depolarization.

An aspect of the method includes receiving electronic information regarding medications administered by a device performing the method.

An aspect of the method includes receiving the medication agent by the device.

An aspect of the method includes receiving the medication dose by the device.

An aspect of the method includes producing an alarm after detection of unintentional recurarization.

An aspect of the method includes triggering an infusion device to administer a reversal agent after detection of unintentional recurarization.

An aspect of the method includes measuring NMB depth and predicting NMB recovery events.

An aspect of the method includes identifying definable points of interest on recovery curves (“landmarks”).

An aspect of the method includes identifying the onset of recovery (“ON”).

An aspect of the method includes identifying the transition point from slow recovery to rapid (“recovery point”).

An aspect of the method includes identifying the point where the quantified muscle response reaches 25% of the maximal muscle response (“EC25”).

An aspect of the method includes identifying the point where the quantified muscle response reaches 50% of the maximal muscle response (“EC50”).

An aspect of the method includes identifying the point where the quantified muscle response reaches 75% of the maximal muscle response (“EC75”).

An aspect of the method includes identifying the transition point from rapid to plateauing recovery (“plateau point”).

An aspect of the method includes identifying the point where recovery first reaches a point deemed to be complete recovery (“OFF”).

An aspect of the method includes estimating a quantified maximal muscle response (Cmax).

An aspect of the method includes using a formula derived from the Hill fractional effect equation to calculate a maximal response in agonist/antagonist systems.

An aspect of the method includes the agonist being acetylcholine released as part of neuromuscular junction transmission and the antagonist being a competitive nondepolarizing neuromuscular blocking agent.

An aspect of the method includes improving the estimates of Cmax via calculations across more than one paired set of data points.

An aspect of the method includes excluding outlying results from the calculations.

An aspect of the method includes applying one or more measures of central tendency to results.

An aspect of the method includes utilizing data found to be adhering to the Hill equation for the calculations.

An aspect of the method includes, based on populations of patients, associating proportions of full recovery with identified points or segments of the recovery curves such that observed data at so-identified portions of the recovery curve allows an estimate of Cmax for individuals.

An aspect of the method includes refining the Cmax estimate as the value that produces the least error between predictive maps of recovery and the observed data.

An aspect of the method includes refining the Cmax estimate using the value that produces the greatest measure of linearity in plots that produce a line when data adheres to the Hill equation.

An aspect of the method includes obtaining a measurement of a level of NMB.

An aspect of the method includes C1/Cmax (C1%) measuring the NMB level.

An aspect of the method includes C2/Cmax (C2%) measuring the NMB level.

An aspect of the method includes C3/Cmax (C3%) measuring the NMB level.

An aspect of the method includes C4/Cmax (C4%) measuring the NMB level.

An aspect of the method includes combinations of C1%, C2%, C3%, and C4% values measuring the NMB level.

An aspect of the method includes the average of C1% and C4% measuring the NMB level.

An aspect of the method includes the observed stage of recovery for an individual providing a measure of the NMB level based on a previously determined average NMB level for each stage across a population.

An aspect of the method includes identifying a stage of recovery and applying its associated previously determined average Cx % for that stage to estimate Cmax.

An aspect of the method includes borders of each stage being determined by predefined landmarks on each curve.

An aspect of the method includes determining stages by visual inspection of the plots of curves.

An aspect of the method includes detecting recurarization.

An aspect of the method includes determining predictions of unobserved portions of the recovery curves to produce recovery maps.

An aspect of the method includes using identified landmarks to predict the remaining unobserved portions of the recovery curves.

An aspect of the method includes using pharmacokinetic/pharmacodynamic (PK/PD) modeling to create a recovery map.

An aspect of the method includes translating points on the recovery curve that are known to be associated with a given receptor occupancy to an effective concentration of NMB agent (“[N]”).

An aspect of the method includes relating [N₀] where receptor occupancy is known to [N] at other time points via first order kinetics equation [N]=[N₀]e^{-kt} where k is the rate constant and t is the time after [N₀].

An aspect of the method includes estimating the rate constant (k) by assessing the time between two or more points of estimated concentration.

An aspect of the method includes refining the rate constant (k) to the value that produces the least error between predictive maps of recovery and the observed data.

An aspect of the method includes solving the Hill fractional effect equation for all time points of interest.

An aspect of the method includes estimating the Hill exponent (γ).

An aspect of the method includes estimating the Hill exponent (γ) specific to each ordinal recovery curve.

An aspect of the method includes estimating the Hill exponent (γ) for an ordinal recovery curve using two distinct points on that curve for which the fractional effect is known or estimated.

An aspect of the method includes estimating the Hill exponent (γ) using a single point where the fractional effect is known on a recovery curve for which the [N] associated with EC50 is known or estimated.

An aspect of the method includes estimating the Hill exponent (γ) using the slope of the line resulting from a plot of logit(Cx %) vs ln[N] where logit(Cx %) is equal to ln[Cx %/(1−Cx %)].

An aspect of the method includes estimating the Hill exponent (γ) from the maximum slope of the Cx % vs time plot.

An aspect of the method includes refining the Hill exponent (γ) as the value that produces the least error between predictive maps of recovery and the observed data.

An aspect of the method includes deriving linear plots from the Hill fractional effect equation.

An aspect of the method includes identifying adherence to the Hill equation by portions of the plot showing linearity.

An aspect of the method includes using loss of linearity of the plot to signal deviation from adherence to the Hill equation and, therefore, deviation from the expected recovery pattern.

An aspect of the method includes plotting logit(Cx %) for observed data vs. time.

An aspect of the method includes defining a line across portions of the plot demonstrating sufficient linearity as logit(Cx %)=mt+b where m is the slope of the so-defined line and b is the y-intercept.

An aspect of the method includes generating the logit recovery map using Cx=Cmax*e^{mt+b}/(e^{mt+b}+1) for all points Cx.

An aspect of the method includes refining m and b as the values that produce the least error between predictive maps of recovery and the observed data.

An aspect of the method includes plotting logit(Cx %) vs. ln[N] for observed data vs. time.

An aspect of the method includes plotting Cx/Cy vs. Cx/Cmax for observed data where x and y denote an ordinal CMAP number at a coincident time and where x>y.

An aspect of the method includes optimizing estimated parameters used in constructing recovery maps to provide the closest fit of the map to the observed data.

An aspect of the method includes adjusting parameters to minimize least squares differences with the observed data.

An aspect of the method includes adjusting parameters to maximize linearity measures for plots expected to be linear.

An aspect of the method includes adjusting parameters to best match a recovery map that is determined to be preferred at the time.

An aspect of the method includes using artificial intelligence to optimize parameters to provide the best fit of recovery maps to observed data.

An aspect of the method includes using machine learning to optimize parameters to provide the best fit of recovery maps to observed data.

An aspect of the method includes using an aggregate recovery map to predict unobserved recovery events.

An aspect of the method includes predicting timing of specified recovery events.

An aspect of the method includes determining a recovery status at a specified time.

An aspect of the method includes using the aggregate recovery map to predict NMB agent dosing.

An aspect of the method includes predicting the recovery status following a specified dose of a NMB agent.

An aspect of the method includes estimating a dose of a NMB agent such that a specified recovery event occurs at a specified time.

An aspect of the method includes estimating the patient's volume of distribution when the aggregate recovery map is used to predict NMB agent dosing.

An aspect of the method includes calculating a patient-specific volume of distribution based on the patient's prior response to NMB agent dosing when the aggregate recovery map is used to predict NMB agent dosing.

An aspect of the method includes assessing new data for deviation from its predicted recovery status.

An aspect of the method includes identifying deviation from the predicted path based on data falling outside expected limits of variability.

An aspect of the method includes identifying deviation from the predicted path based on nonlinear behavior for new data within a linearity measure.

An aspect of the method includes identifying deviation from the predicted path based on an abrupt rise in ordinal CMAPs inconsistent with the stage of NMB recovery.

An aspect of the method includes identifying deviation from the predicted path based on abrupt decline in ordinal CMAPs inconsistent with the stage of NMB recovery.

An aspect of the method includes model compensation for subtle gradual deviation from the originally predicted path.

An aspect of the method includes identifying trends in data by variations in recovery maps across cycles of recovery.

An aspect of the method includes more recent data being weighted more in the analysis than older data.

An aspect of the method includes the recovery map directing administration of NMB agents.

An aspect of the method includes calculating a patient-specific effective volume of distribution for an NMB agent.

An aspect of the method includes storing all relevant results of NMB analysis for use in future medical procedures requiring NMB for the same patient.

An aspect of the method includes obtaining early estimation of Cmax based on Cx % values associated with early landmarks in follow on medical procedures.

An aspect of the method includes translating calculated parameters in recovery map creation for use in follow on medical procedures.

An aspect of the method includes translating dosing parameters for use in follow on medical procedures.

An aspect of the method includes relating measurement of NMB across different muscles to determine a systemic level of NMB.

An aspect of the method includes characterizing differences in NMB recovery patterns between different muscles by simultaneous measurements over a population of patients.

An aspect of the method includes using transitive properties across muscles for the NMB depth for prediction of equivalent findings between any two muscles that have been mapped to the reference muscle.

An aspect of the method includes describing the systemic level of NMB in terms of a reference muscle's expected response at any given NMB depth level.

An aspect of the method includes mapping muscle function to the systemic level of NMB.

An aspect of the method includes mapping the suppressive effect of NMB on clinical tests generating muscle responses to the systemic level of NMB.

An aspect of the method includes converting tests generating muscle responses in the presence of NMB to the estimated equivalent result when the NMB effect is removed.

An aspect of the method includes characterizing the probability of a specified degree of loss of clinical function using the reference muscle's Cx % measure across the NMB spectrum.

An aspect of the method includes predictions of the impact of NMB on functional tests being further stratified by the initial signal quality which may include absolute quantities/amplitudes in the absence of NMB, preceding variability, and signal-to-noise ratio.

An aspect of the method includes characterizing the probability of a specified degree of loss of muscle tested quantity using the reference muscle's Cx % measure across the NMB spectrum.

An aspect of the method includes predictions of the impact of NMB on muscle testing being further stratified by the initial signal quality which may include absolute quantities/amplitudes in the absence of NMB, preceding variability, and signal-to-noise ratio.

An aspect of the method includes using the C4% for determination of return of respiratory function sufficient to allow removal of ventilatory support.

An aspect of the method includes using the average of C1% and C4% for determination of return of respiratory function sufficient to allow removal of ventilatory support.

An aspect of the method includes using mechanical measures of NMB to generate a map of the systemic level of NMB based on a mechanical response in the reference muscles.

An aspect of the method includes using mechanical measures incorporated into their equivalent value within an EMG-determined system of systemic level of NMB.

An aspect of the method includes using mechanical measures.

An aspect of the method includes providing correction for skew related to mechanical measures.

An aspect of the method includes converting mechanical measures to EMG-equivalents.

Aspects of the embodiments may include a device to estimate a degree of muscle recovery from non-depolarizing neuromuscular blocking agents comprising: an electrical stimulator to apply a repetitive nerve stimulation to a motor nerve; and a processor configured to perform operations including: quantitating each muscle response resulting from repetitive stimulation; tracking a recovery pattern of each ordinal muscle response versus time; identifying targeted portions of recovery curves; predicting an individual's elimination rate for an NMB agent; predicting a maximal muscle response upon recovery; predicting full recovery curves for each ordinal muscle response; utilizing a predicted muscle response quantity at full recovery to determine a degree of recovery for each ordinal muscle response at a time of testing; utilizing predicted recovery curves to predict a timing of any future degree of recovery of interest; updating predictions over time as further patient-specific data is gathered; and providing recommendations guiding clinical decision making.

Having described preferred embodiments of a new and improved system, method, and computer program product for the assessment and prediction of recovery from pharmacologic neuromuscular blocking agents, variations and changes will be suggested to those skilled in the art in view of the teachings set forth herein. It is therefore to be understood that all such variations, modifications and changes are believed to fall within the scope of present invention embodiments as defined by the appended claims.

What is claimed is:

1. A method to estimate a degree of muscle recovery from non-depolarizing neuromuscular blocking agents comprising:
tracking, via at least one processor, a recovery pattern of a muscle based on values of muscle responses to a series of stimuli, wherein the values of the muscle responses to stimuli over time produce points indicating recovery curves of the muscle for the series of stimuli;
determining, via the at least one processor, points of interest based on the recovery curves;
predicting, via the at least one processor, a recovery value for muscle response for recovery from a neuromuscular blocking agent based on the values of muscle responses;
estimating, via the at least one processor, a degree of effect produced by the neuromuscular blocking agent based on the recovery value;
generating, via the at least one processor, a recovery map of the recovery curves for the muscle responses by predicting contiguous recovery curves across a time of interest based on at least two selected from a group of the values of the muscle responses, the points of interest, and the recovery value; and
determining, via the at least one processor, a dosage for the neuromuscular blocking agent enabling the muscle to attain recovery at a certain time based on the recovery map.

2. The method of claim 1, wherein the points of interest include a point indicating onset of recovery, a point indicating completion of recovery, a point indicating transition to a plateau, a point indicating transition to rapid recovery, and one or more points corresponding to percentages of the recovery value.

3. The method of claim 1, wherein the recovery value is determined based on a Hill fractional effect formula.

4. The method of claim 1, wherein a measure of the degree of effect of the neuromuscular blocking agent is based on one or more ratios of a value of an ordered muscle response over the recovery value.

5. The method of claim 1, wherein the recovery map is generated based on pharmacokinetic/pharmacodynamic (PK/PD) modeling.

6. The method of claim 1, wherein the recovery map is generated based on parameters derived from linearized values of muscle responses to a stimulation signal over time.

7. The method of claim 1, wherein parameters used to generate the recovery map are optimized to provide a closest fit of the recovery map to observed data.

8. The method of claim 1, further comprising:
determining, via the at least one processor, a systemic amount of the neuromuscular blocking agent based on measurement of the neuromuscular blocking agent across different muscles.

9. The method of claim 1, further comprising:
updating, via the at least one processor, the recovery map based on detection of a reversal agent failing to cause reversal of the neuromuscular blocking agent.

10. The method of claim 1, further comprising:
generating, via the at least one processor, an alarm based on detection of reversal of the neuromuscular blocking agent by a reversal agent and unintentional recurarization.

11. A system to estimate a degree of muscle recovery from non-depolarizing neuromuscular blocking agents comprising:
one or more memories; and
at least one processor coupled to the one or more memories and configured to perform operations including:
tracking a recovery pattern of a muscle based on values of muscle responses to a series of stimuli, wherein the values of the muscle responses to stimuli over time produce points indicating recovery curves of the muscle for the series of stimuli;
determining points of interest based on the recovery curves;
predicting a recovery value for muscle response for recovery from a neuromuscular blocking agent based on the values of muscle responses;
estimating a degree of effect produced by the neuromuscular blocking agent based on the recovery value;
generating a recovery map of the recovery curves for the muscle responses by predicting contiguous recovery curves across a time of interest based on at least two selected from a group of the values of the muscle responses, the points of interest, and the recovery value; and
determining a dosage for the neuromuscular blocking agent enabling the muscle to attain recovery at a certain time based on the recovery map.

12. The system of claim 11, wherein the points of interest include a point indicating onset of recovery, a point indicating completion of recovery, a point indicating transition to a plateau, a point indicating transition to rapid recovery, and one or more points corresponding to percentages of the recovery value.

13. The system of claim 11, wherein the recovery value is determined based on a Hill fractional effect formula.

14. The system of claim 11, wherein a measure of the degree of effect of the neuromuscular blocking agent is based on one or more ratios of a value of an ordered muscle response over the recovery value.

15. The system of claim 11, wherein the recovery map is generated based on pharmacokinetic/pharmacodynamic (PK/PD) modeling.

16. The system of claim 11, wherein the recovery map is generated based on parameters derived from linearized values of muscle responses to a stimulation signal over time.

17. The system of claim 11, wherein parameters used to generate the recovery map are optimized to provide a closest fit of the recovery map to observed data.

18. The system of claim 11, wherein the at least one processor is configured to perform further operations comprising:
determining a systemic amount of the neuromuscular blocking agent based on measurement of the neuromuscular blocking agent across different muscles.

19. The system of claim 11, wherein the at least one processor is configured to perform further operations comprising:
updating the recovery map based on detection of a reversal agent failing to cause reversal of the neuromuscular blocking agent.

20. The system of claim 11, wherein the at least one processor is configured to perform further operations comprising:
generating an alarm based on detection of reversal of the neuromuscular blocking agent by a reversal agent and unintentional recurarization.

21. A computer program product comprising a non-transitory computer readable medium encoded with instructions that, when executed by at least one processor, cause the at least one processor to perform operations including:
tracking a recovery pattern of a muscle based on values of muscle responses to a series of stimuli, wherein the values of the muscle responses to stimuli over time produce points indicating recovery curves of the muscle for the series of stimuli;
determining points of interest based on the recovery curves;
predicting a recovery value for muscle response for recovery from a neuromuscular blocking agent based on the values of muscle responses;
estimating a degree of effect produced by the neuromuscular blocking agent based on the recovery value;
generating a recovery map of the recovery curves for the muscle responses by predicting contiguous recovery curves across a time of interest based on at least two selected from a group of the values of the muscle responses, the points of interest, and the recovery value; and
determining a dosage for the neuromuscular blocking agent enabling the muscle to attain recovery at a certain time based on the recovery map.

22. The computer program product of claim 21, wherein the points of interest include a point indicating onset of recovery, a point indicating completion of recovery, a point indicating transition to a plateau, a point indicating transition to rapid recovery, and one or more points corresponding to percentages of the recovery value.

23. The computer program product of claim 21, wherein the recovery value is determined based on a Hill fractional effect formula.

24. The computer program product of claim 21, wherein a measure of the degree of effect of the neuromuscular blocking agent is based on one or more ratios of a value of an ordered muscle response over the recovery value.

25. The computer program product of claim 21, wherein the recovery map is generated based on pharmacokinetic/pharmacodynamic (PK/PD) modeling.

26. The computer program product of claim 21, wherein the recovery map is generated based on parameters derived from linearized values of muscle responses to a stimulation signal over time.

27. The computer program product of claim 21, wherein parameters used to generate the recovery map are optimized to provide a closest fit of the recovery map to observed data.

28. The computer program product of claim 21, wherein the instructions cause the at least one processor to perform further operations including:
determining a systemic amount of the neuromuscular blocking agent based on measurement of the neuromuscular blocking agent across different muscles.

29. The computer program product of claim 21, wherein the instructions cause the at least one processor to perform further operations including:
updating the recovery map based on detection of a reversal agent failing to cause reversal of the neuromuscular blocking agent.

30. The computer program product of claim 21, wherein the instructions cause the at least one processor to perform further operations including:
generating an alarm based on detection of reversal of the neuromuscular blocking agent by a reversal agent and unintentional recurarization.
